(19) United States
(12) Patent Application Publication
(10) Pub. No.: US 2017/0119262 A1
(43) Pub. Date: May 4, 2017

(54) MOBILE TERMINAL

(71) Applicant: LG ELECTRONICS INC., Seoul (KR)

(72) Inventors: Hongjo SHIM, Seoul (KR); Seonghyok KIM, Seoul (KR)

(73) Assignee: LG ELECTRONICS INC., Seoul (KR)

(21) Appl. No.: 15/159,569

(22) Filed: May 19, 2016

(30) Foreign Application Priority Data
Oct. 28, 2015 (KR) 10-2015-0150484

(51) Int. Cl. A61B 5/021; A61B 5/0408; A61B 5/00; H04W 4/00; H04B 1/3827

(52) U.S. Cl. CPC A61B 5/02108; H04W 4/005; H04W 4/001; H04B 1/385; A61B 5/6898; A61B 5/0013; A61B 5/7405; A61B 5/0408; A61B 5/0006; A61B 5/681

(57) ABSTRACT

A mobile terminal includes: a terminal body; a first sensor part formed on an external surface of the terminal body, having a light emitting portion and a light receiving portion, and configured to collect pulse wave information; a second sensor part disposed on one region of the terminal body, and configured to collect an additional bio signal; and a controller configured to calculate a blood pressure based on the pulse wave information and the additional bio signal.

### CROSS-REFERENCE TO RELATED APPLICATION

Pursuant to 35 U.S.C. §119(a), this application claims the benefit of earlier filing date and right of priority to Korean Application No. 10-2015-0150484, filed on Oct. 28, 2015, the contents of which is incorporated by reference herein in its entirety.

### TECHNICAL FIELD

The present invention relates to a mobile terminal capable of collecting user's bio information.

### BACKGROUND ART

Terminals may be generally classified as mobile/portable terminals or stationary terminals according to their mobility. Mobile terminals may also be classified as handheld terminals or vehicle mounted terminals according to whether or not a user can directly carry the terminal.

Mobile terminals have become increasingly more functional. Examples of such functions include data and voice communications, capturing images and video via a camera, recording audio, playing music files via a speaker system, and displaying images and video on a display. Some mobile terminals include additional functionality which supports game playing, while other terminals are configured as multimedia players. More recently, mobile terminals have been configured to receive broadcast and multicast signals which permit viewing of content such as videos and television programs.

Various attempts have been made to implement complicated functions in such a multimedia device by means of hardware or software. Recently, research on various functions to collect bio information by a sensor, etc. included in a wearable-type mobile terminal mounted to a human body is ongoing actively. However, in case of mounting an additional sensing module for collecting bio information, the mobile terminal may have a large weight, and a user should contact part of his or her body onto the sensing module. This may cause user's inconvenience.

### DISCLOSURE OF THE INVENTION

Therefore, an object of the present invention is to provide a mobile terminal capable of measuring a blood pressure.

To achieve these and other advantages and in accordance with the purpose of the present invention, as embodied and broadly described herein, there is provided a mobile terminal, including: a terminal body; a first sensor part formed on an external surface of the terminal body, having a light emitting portion and a light receiving portion, and configured to collect pulse wave information; a second sensor part disposed on one region of the terminal body, and configured to collect an additional bio signal; and a controller configured to calculate a blood pressure based on the pulse wave information and the additional bio signal.

In an embodiment of the present invention, the second sensor part may correspond to a pressure sensor for sensing an external force, and the additional bio signal may correspond to a pressure change sensed while the pulse wave information is collected by the first sensor part. Thus, the mobile terminal may measure a blood pressure based on a pulse wave change due to a pressure change.

In an embodiment of the present invention, the second sensor part may include first and second electrode portions disposed on different regions of the terminal body, and generating a potential difference. The controller may collect electrocardiogram information based on the potential difference. Thus, the mobile terminal may measure a pressure based on the electrocardiogram information and pulse wave information.

In an embodiment of the present invention, the mobile terminal may further include a band for fixing the terminal body to a user's wrist, and the second electrode portion may be disposed on one region of the band. The band may further include a flexible printed circuit board for electrically connecting the second electrode portion to the terminal body. In case of the watch-type mobile terminal, a sensor for collecting pulse wave information and electrocardiogram information is mounted to a region of the mobile terminal which contacts a human body when the user wears the mobile terminal. Accordingly, the watch-type mobile terminal can easily measure a blood pressure.

### Effects of the Present Invention

In the mobile terminal according to an embodiment of the present invention, a signal input unit for inputting a control signal by a pressurization structure includes a PPG sensor and a pressure sensor. Thus, information for calculating a blood pressure may be obtained without an additional space for the PPG sensor and the pressure sensor. Further, since the PPG sensor and the pressure sensor are integrally formed with the signal input unit, bio information for calculating a blood pressure may be obtained while a specific function is being executed. Thus, an additional sensor module for measuring a blood pressure is not required.

Further, an electrode unit, part of an ECG module for collecting electrocardiogram information is disposed close to electronic components of the mobile terminal. Thus, an additional sensing module is not required, and the electrode unit is not covered by a case mounted to the terminal body. Further, in case of using the electronic components, bio information may be collected.

### BRIEF DESCRIPTION OF THE DRAWINGS

FIG. 1A is a block diagram of a mobile terminal according to the present invention;

FIGS. 1B and 1C are conceptual views illustrating an example of a mobile terminal according to the present invention, which are viewed from different directions;

FIG. 2A is a sectional view illustrating a structure of a sensing module capable of collecting bio-information;

FIG. 2B is a conceptual view illustrating a pressure sensor;

FIG. 3 is a conceptual view illustrating a sensing module according to an embodiment of the present invention;

FIGS. 4A and 4B are conceptual views illustrating a sensing module capable of sensing a fingerprint according to an embodiment of the present invention;

FIG. 4C is a view illustrating a pulse wave measured while a pressure is applied;

FIGS. 5A to 5C are conceptual views illustrating a sensing module according to another embodiment of the present invention;

FIG. 6 is a conceptual view illustrating a sensing module according to still another embodiment of the present invention;

FIGS. 7A and 7B are conceptual views illustrating an ECG module according to an embodiment of the present invention;

FIGS. 7C and 7D are conceptual views illustrating an electrode unit disposed close to an audio output module;

FIGS. 8A and 8B are conceptual views illustrating an electrode unit according to another embodiment of the present invention;

FIG. 9A is a conceptual view illustrating a control method of measuring a blood pressure;

FIG. 9B is a conceptual view illustrating a method of measuring a blood pressure based on information on an electrocardiogram (ECG) and a pulse wave;

FIG. 10A is a block diagram illustrating a mobile terminal according to another embodiment of the present invention;

FIG. 10B is a conceptual view illustrating an example of a mobile terminal according to the present invention, which is viewed from one direction;

FIG. 11A is a conceptual view illustrating a sensing module for measuring a blood pressure according to an embodiment of the present invention;

FIG. 11B is a conceptual view illustrating components of the sensing module;

FIG. 11C is a conceptual view illustrating a control method of outputting guide information while a blood pressure is being measured;

FIGS. 12A to 12D are conceptual views illustrating a mobile terminal including an electrode unit for measuring an electrocardiogram (ECG); and

FIG. 13 is a conceptual view illustrating a control method of providing a result on a blood pressure measured according to an embodiment of the present invention.

### MODES FOR CARRYING OUT THE PREFERRED EMBODIMENTS

Description will now be given in detail according to exemplary embodiments disclosed herein, with reference to the accompanying drawings. For the sake of brief description with reference to the drawings, the same or equivalent components may be provided with the same or similar reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

A terminal in the present description may include a mobile terminal such as a portable phone, a smart phone, a notebook computer, a digital broadcasting terminal, Personal Digital Assistants (PDA), Portable Multimedia Player (PMP), a navigation system, a slate PC, a tablet PC, an ultra book, a wearable device (e.g., smart watch), a glass-type terminal (e.g., smart glass), a head mounted display (HMD), etc.

However, it will be obvious to those skilled in the art that the present invention may be also applicable to a fixed terminal such as a digital TV, a desktop computer and a digital signage, except for specific configurations for mobility.

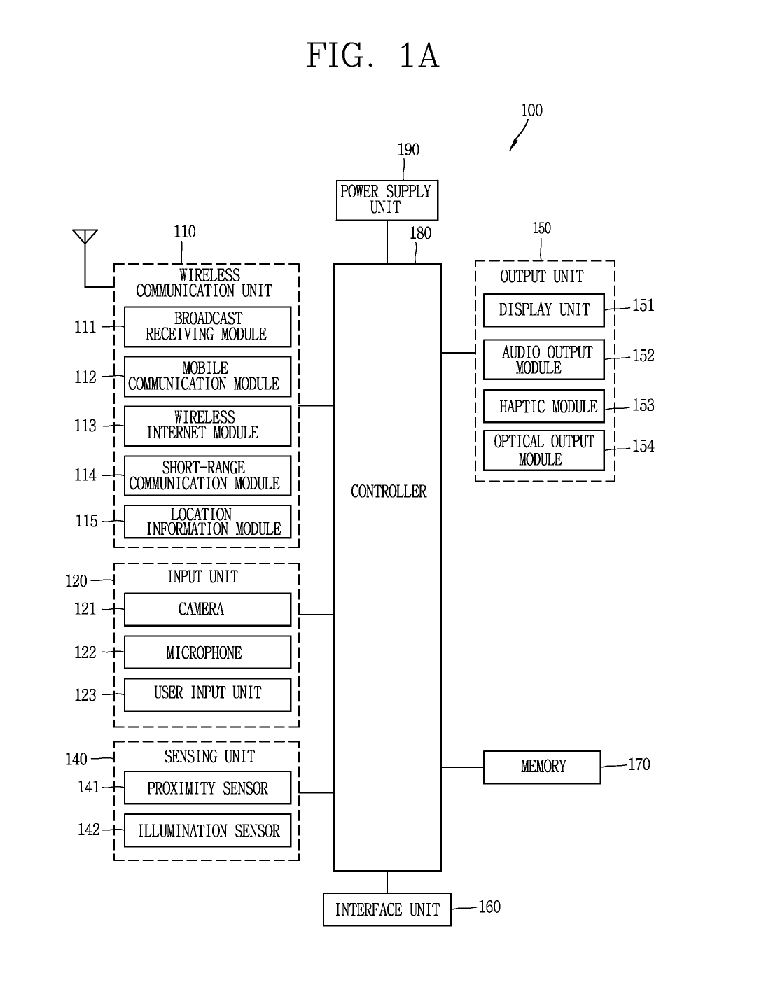

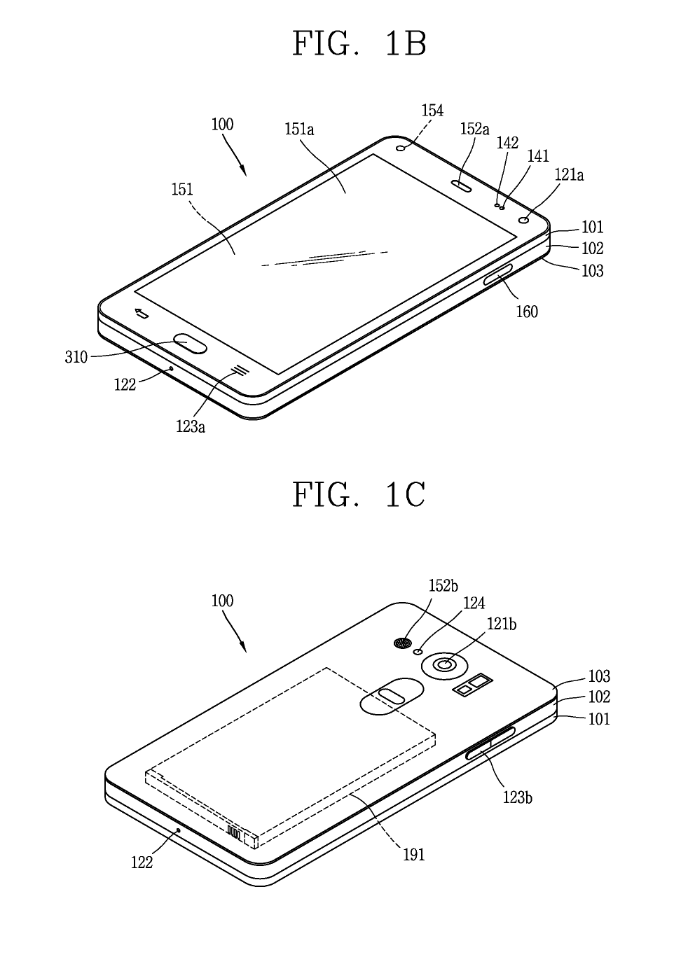

Reference is now made to FIGS. 1A-1C, where FIG. 1A is a block diagram of a mobile terminal in accordance with the present disclosure, and FIGS. 1B and 1C are conceptual views of one example of the mobile terminal, viewed from different directions.

The mobile terminal 100 is shown having components such as a wireless communication unit 110, an input unit 120, a sensing unit 140, an output unit 150, an interface unit 160, a memory 170, a controller 180, and a power supply unit 190. It is understood that implementing all of the illustrated components of FIG. 1A is not a requirement, and that greater or fewer components may alternatively be implemented.

Referring now to FIG. 1A, the mobile terminal 100 is shown having wireless communication unit 110 configured with several commonly implemented components. For instance, the wireless communication unit 110 typically includes one or more components which permit wireless communication between the mobile terminal 100 and a wireless communication system or network within which the mobile terminal is located.

The wireless communication unit 110 typically includes one or more modules which permit communications such as wireless communications between the mobile terminal 100 and a wireless communication system, communications between the mobile terminal 100 and another mobile terminal, communications between the mobile terminal 100 and an external server. Further, the wireless communication unit 110 typically includes one or more modules which connect the mobile terminal 100 to one or more networks. To facilitate such communications, the wireless communication unit 110 includes one or more of a broadcast receiving module 111, a mobile communication module 112, a wireless Internet module 113, a short-range communication module 114, and a location information module 115.

The input unit 120 includes a camera 121 for obtaining images or video, a microphone 122, which is one type of audio input device for inputting an audio signal, and a user input unit 123 (for example, a touch key, a push key, a mechanical key, a soft key, and the like) for allowing a user to input information. Data (for example, audio, video, image, and the like) is obtained by the input unit 120 and may be analyzed and processed by controller 180 according to device parameters, user commands, and combinations thereof.

The sensing unit 140 is typically implemented using one or more sensors configured to sense internal information of the mobile terminal, the surrounding environment of the mobile terminal, user information, and the like. For example, in FIG. 1A, the sensing unit 140 is shown having a proximity sensor 141 and an illumination sensor 142. If desired, the sensing unit 140 may alternatively or additionally include other types of sensors or devices, such as a touch sensor, an acceleration sensor, a magnetic sensor, a G-sensor, a gyroscope sensor, a motion sensor, an RGB sensor, an infrared (IR) sensor, a finger scan sensor, a ultrasonic sensor, an optical sensor (for example, camera 121), a microphone 122, a battery gauge, an environment sensor (for example, a barometer, a hygrometer, a thermometer, a radiation detection sensor, a thermal sensor, and a gas sensor, among others), and a chemical sensor (for example, an electronic nose, a health care sensor, a biometric sensor, and the like), to name a few. The mobile terminal 100 may be configured to utilize information obtained from sensing unit 140, and in particular, information obtained from one or more sensors of the sensing unit 140, and combinations thereof.

The output unit 150 is typically configured to output various types of information, such as audio, video, tactile output, and the like. The output unit 150 is shown having a display unit 151, an audio output module 152, a haptic module 153, and an optical output module 154.

The display unit 151 may have an inter-layered structure or an integrated structure with a touch sensor in order to facilitate a touch screen. The touch screen may provide an output interface between the mobile terminal 100 and a user, as well as function as the user input unit 123 which provides an input interface between the mobile terminal 100 and the user.

The interface unit 160 serves as an interface with various types of external devices that can be coupled to the mobile terminal 100. The interface unit 160, for example, may include any of wired or wireless ports, external power supply ports, wired or wireless data ports, memory card ports, ports for connecting a device having an identification module, audio input/output (I/O) ports, video I/O ports, earphone ports, and the like. In some cases, the mobile terminal 100 may perform assorted control functions associated with a connected external device, in response to the external device being connected to the interface unit 160.

The memory 170 is typically implemented to store data to support various functions or features of the mobile terminal 100. For instance, the memory 170 may be configured to store application programs executed in the mobile terminal 100, data or instructions for operations of the mobile terminal 100, and the like. Some of these application programs may be downloaded from an external server via wireless communication. Other application programs may be installed within the mobile terminal 100 at time of manufacturing or shipping, which is typically the case for basic functions of the mobile terminal 100 (for example, receiving a call, placing a call, receiving a message, sending a message, and the like). It is common for application programs to be stored in the memory 170, installed in the mobile terminal 100, and executed by the controller 180 to perform an operation (or function) for the mobile terminal 100.

The controller 180 typically functions to control overall operation of the mobile terminal 100, in addition to the operations associated with the application programs. The controller 180 may provide or process information or functions appropriate for a user by processing signals, data, information and the like, which are input or output by the various components depicted in FIG. 1A, or activating application programs stored in the memory 170. As one example, the controller 180 controls some or all of the components illustrated in FIGS. 1A-1C according to the execution of an application program that have been stored in the memory 170.

The power supply unit 190 can be configured to receive external power or provide internal power in order to supply appropriate power required for operating elements and components included in the mobile terminal 100. The power supply unit 190 may include a battery, and the battery may be configured to be embedded in the terminal body, or configured to be detachable from the terminal body.

At least some of the above components may operate in a cooperating manner, so as to implement an operation or a control method of a glass type terminal according to various embodiments to be explained later. The operation or the control method of the glass type terminal may be implemented on the glass type terminal by driving at least one application program stored in the memory 170.

Referring still to FIG. 1A, various components depicted in this figure will now be described in more detail. Regarding the wireless communication unit 110, the broadcast receiving module 111 is typically configured to receive a broadcast signal and/or broadcast associated information from an external broadcast managing entity via a broadcast channel. The broadcast channel may include a satellite channel, a terrestrial channel, or both. In some embodiments, two or more broadcast receiving modules 111 may be utilized to facilitate simultaneously receiving of two or more broadcast channels, or to support switching among broadcast channels.

The mobile communication module 112 can transmit and/or receive wireless signals to and from one or more network entities. Typical examples of a network entity include a base station, an external mobile terminal, a server, and the like. Such network entities form part of a mobile communication network, which is constructed according to technical standards or communication methods for mobile communications (for example, Global System for Mobile Communication (GSM), Code Division Multi Access (CDMA), CDMA2000(Code Division Multi Access 2000), EV-DO(Enhanced Voice-Data Optimized or Enhanced Voice-Data Only), Wideband CDMA (WCDMA), High Speed Downlink Packet access (HSDPA), HSUPA(High Speed Uplink Packet Access), Long Term Evolution (LTE), LTE-A(Long Term Evolution-Advanced), and the like).

Examples of wireless signals transmitted and/or received via the mobile communication module 112 include audio call signals, video (telephony) call signals, or various formats of data to support communication of text and multimedia messages.

The wireless Internet module 113 is configured to facilitate wireless Internet access. This module may be internally or externally coupled to the mobile terminal 100. The wireless Internet module 113 may transmit and/or receive wireless signals via communication networks according to wireless Internet technologies.

Examples of such wireless Internet access include Wireless LAN (WLAN), Wireless Fidelity (Wi-Fi), Wi-Fi Direct, Digital Living Network Alliance (DLNA), Wireless Broadband (WiBro), Worldwide Interoperability for Microwave Access (WiMAX), High Speed Downlink Packet Access (HSDPA), Long Term Evolution (LTE), and the like. The wireless Internet module 113 may transmit/receive data according to one or more of such wireless Internet technologies, and other Internet technologies as well.

In some embodiments, when the wireless Internet access is implemented according to, for example, WiBro, HSDPA,HSUPA, GSM, CDMA, WCDMA, LTE, LTE-A and the like, as part of a mobile communication network, the wireless Internet module 113 performs such wireless Internet access. As such, the Internet module 113 may cooperate with, or function as, the mobile communication module 112.

The short-range communication module 114 is configured to facilitate short-range communications. Suitable technologies for implementing such short-range communications include BLUETOOTH™, Radio Frequency IDentification (RFID), Infrared Data Association (IrDA), Ultra-WideBand (UWB), ZigBee, Near Field Communication (NFC), Wireless-Fidelity (Wi-Fi), Wi-Fi Direct, Wireless USB(Wireless Universal Serial Bus), and the like. The short-range communication module 114 in general supports wireless communications between the mobile terminal 100 and a wireless communication system, communications between the mobile terminal 100 and another mobile terminal 100, or communications between the mobile terminal and a network where another mobile terminal 100 (or an external server) is located, via wireless area networks. One example of the wireless area networks is a wireless personal area networks.

In some embodiments, another mobile terminal (which may be configured similarly to mobile terminal 100) may be a wearable device, for example, a smart watch, a smart glass or a head mounted display (HMD), which is able to exchange data with the mobile terminal 100 (or otherwise cooperate with the mobile terminal 100). The short-range communication module 114 may sense or recognize the wearable device, and permit communication between the wearable device and the mobile terminal 100. In addition, when the sensed wearable device is a device which is authenticated to communicate with the mobile terminal 100, the controller 180, for example, may cause transmission of data processed in the mobile terminal 100 to the wearable device via the short-range communication module 114. Hence, a user of the wearable device may use the data processed in the mobile terminal 100 on the wearable device. For example, when a call is received in the mobile terminal 100, the user may answer the call using the wearable device. Also, when a message is received in the mobile terminal 100, the user can check the received message using the wearable device.

The location information module 115 is generally configured to detect, calculate, derive or otherwise identify a position of the mobile terminal. As an example, the location information module 115 includes a Global Position System (GPS) module, a Wi-Fi module, or both. If desired, the location information module 115 may alternatively or additionally function with any of the other modules of the wireless communication unit 110 to obtain data related to the position of the mobile terminal.

As one example, when the mobile terminal uses a GPS module, a position of the mobile terminal may be acquired using a signal sent from a GPS satellite. As another example, when the mobile terminal uses the Wi-Fi module, a position of the mobile terminal can be acquired based on information related to a wireless access point (AP) which transmits or receives a wireless signal to or from the Wi-Fi module.

The input unit 120 may be configured to permit various types of input to the mobile terminal 120. Examples of such input include audio, image, video, data, and user input. Image and video input is often obtained using one or more cameras 121. Such cameras 121 may process image frames of still pictures or video obtained by image sensors in a video or image capture mode. The processed image frames can be displayed on the display unit 151 or stored in memory 170. In some cases, the cameras 121 may be arranged in a matrix configuration to permit a plurality of images having various angles or focal points to be input to the mobile terminal 100. As another example, the cameras 121 may be located in a stereoscopic arrangement to acquire left and right images for implementing a stereoscopic image.

The microphone 122 is generally implemented to permit audio input to the mobile terminal 100. The audio input can be processed in various manners according to a function being executed in the mobile terminal 100. If desired, the microphone 122 may include assorted noise removing algorithms to remove unwanted noise generated in the course of receiving the external audio.

The user input unit 123 is a component that permits input by a user. Such user input may enable the controller 180 to control operation of the mobile terminal 100. The user input unit 123 may include one or more of a mechanical input element (for example, a key, a button located on a front and/or rear surface or a side surface of the mobile terminal 100, a dome switch, a jog wheel, a jog switch, and the like), or a touch-sensitive input, among others. As one example, the touch-sensitive input may be a virtual key or a soft key, which is displayed on a touch screen through software processing, or a touch key which is located on the mobile terminal at a location that is other than the touch screen. On the other hand, the virtual key or the visual key may be displayed on the touch screen in various shapes, for example, graphic, text, icon, video, or a combination thereof.

The user input unit 123 may recognize information sensed by the sensing unit 140, as well as by the aforementioned mechanical input means and touch type input means, as information input from a user. Accordingly, the controller 180 can control an operation of the mobile terminal 100 corresponding to the sensed information.

The sensing unit 140 is generally configured to sense one or more of internal information of the mobile terminal, surrounding environment information of the mobile terminal, user information, or the like. The controller 180 generally cooperates with the sending unit 140 to control operation of the mobile terminal 100 or execute data processing, a function or an operation associated with an application program installed in the mobile terminal based on the sensing provided by the sensing unit 140. The sensing unit 140 may be implemented using any of a variety of sensors, some of which will now be described in more detail.

The proximity sensor 141 may include a sensor to sense presence or absence of an object approaching a surface, or an object located near a surface, by using an electromagnetic field, infrared rays, or the like without a mechanical contact. The proximity sensor 141 may be arranged at an inner region of the mobile terminal covered by the touch screen, or near the touch screen.

The proximity sensor 141, for example, may include any of a transmissive type photoelectric sensor, a direct reflective type photoelectric sensor, a mirror reflective type photoelectric sensor, a high-frequency oscillation proximity sensor, a capacitance type proximity sensor, a magnetic type proximity sensor, an infrared rays proximity sensor, and the like. When the touch screen is implemented as a capacitance type, the proximity sensor 141 can sense proximity of a pointer relative to the touch screen by changes of an electromagnetic field, which is responsive to an approach of an object with conductivity. In this case, the touch screen (touch sensor) may also be categorized as a proximity sensor.

The term “proximity touch” will often be referred to herein to denote the scenario in which a pointer is positioned to be proximate to the touch screen without contacting the touch screen. The term “contact touch” will often be referred to herein to denote the scenario in which a pointer makes physical contact with the touch screen. For the position corresponding to the proximity touch of the pointer relative to the touch screen, such position will correspond to a position where the pointer is perpendicular to the touch screen. The proximity sensor 141 may sense proximity touch, and proximity touch patterns (for example, distance, direction, speed, time, position, moving status, and the like).

In general, controller 180 processes data corresponding to proximity touches and proximity touch patterns sensed by the proximity sensor 141, and cause output of visual information on the touch screen. In addition, the controller 180 can control the mobile terminal 100 to execute different operations or process different data according to whether a touch with respect to a point on the touch screen is either a proximity touch or a contact touch.

A touch sensor can sense a touch applied to the touch screen, such as display unit 151, using any of a variety of touch methods. Examples of such touch methods include a resistive type, a capacitive type, an infrared type, and a magnetic field type, among others.

As one example, the touch sensor may be configured to convert changes of pressure applied to a specific part of the display unit 151, or convert capacitance occurring at a specific part of the display unit 151, into electric input signals. The touch sensor may also be configured to sense not only a touched position and a touched area, but also touch pressure and/or touch capacitance. A touch object is generally used to apply a touch input to the touch sensor. Examples of typical touch objects include a finger, a touch pen, a stylus pen, a pointer, or the like.

When a touch input is sensed by a touch sensor, corresponding signals may be transmitted to a touch controller. The touch controller may process the received signals, and then transmit corresponding data to the controller 180. Accordingly, the controller 180 may sense which region on the display unit 151 has been touched. Here, the touch controller may be a component separate from the controller 180, the controller 180, and combinations thereof.

In some embodiments, the controller 180 may execute the same or different controls according to a type of touch object that touches the touch screen or a touch key provided in addition to the touch screen. Whether to execute the same or different control according to the object which provides a touch input may be decided based on a current operating state of the mobile terminal 100 or a currently executed application program, for example.

The touch sensor and the proximity sensor may be implemented individually, or in combination, to sense various types of touches. Such touches includes a short (or tap) touch, a long touch, a multi-touch, a drag touch, a flick touch, a pinch-in touch, a pinch-out touch, a swipe touch, a hovering touch, and the like.

If desired, an ultrasonic sensor may be implemented to recognize position information relating to a touch object using ultrasonic waves. The controller 180, for example, may calculate a position of a wave generation source based on information sensed by an illumination sensor and a plurality of ultrasonic sensors. Since light is much faster than ultrasonic waves, the time for which the light reaches the optical sensor is much shorter than the time for which the ultrasonic wave reaches the ultrasonic sensor. The position of the wave generation source may be calculated using this fact. For instance, the position of the wave generation source may be calculated using the time difference from the time that the ultrasonic wave reaches the sensor based on the light as a reference signal.

The camera 121 typically includes at least one a camera sensor (CCD, CMOS etc.), a photo sensor (or image sensors), and a laser sensor.

Implementing the camera 121 with a laser sensor may allow detection of a touch of a physical object with respect to a 3D stereoscopic image. The photo sensor may be laminated on, or overlapped with, the display device. The photo sensor may be configured to scan movement of the physical object in proximity to the touch screen. In more detail, the photo sensor may include photo diodes and transistors at rows and columns to scan content received at the photo sensor using an electrical signal which changes according to the quantity of applied light. Namely, the photo sensor may calculate the coordinates of the physical object according to variation of light to thus obtain position information of the physical object.

The display unit 151 is generally configured to output information processed in the mobile terminal 100. For example, the display unit 151 may display execution screen information of an application program executing at the mobile terminal 100 or user interface (UI) and graphic user interface (GUI) information in response to the execution screen information.

In some embodiments, the display unit 151 may be implemented as a stereoscopic display unit for displaying stereoscopic images. A typical stereoscopic display unit may employ a stereoscopic display scheme such as a stereoscopic scheme (a glass scheme), an auto-stereoscopic scheme (glassless scheme), a projection scheme (holographic scheme), or the like.

The audio output module 152 is generally configured to output audio data. Such audio data may be obtained from any of a number of different sources, such that the audio data may be received from the wireless communication unit 110 or may have been stored in the memory 170. The audio data may be output during modes such as a signal reception mode, a call mode, a record mode, a voice recognition mode, a broadcast reception mode, and the like. The audio output module 152 can provide audible output related to a particular function (e.g., a call signal reception sound, a message reception sound, etc.) performed by the mobile terminal 100. The audio output module 152 may also be implemented as a receiver, a speaker, a buzzer, or the like.

A haptic module 153 can be configured to generate various tactile effects that a user feels, perceive, or otherwise experience. A typical example of a tactile effect generated by the haptic module 153 is vibration. The strength, pattern and the like of the vibration generated by the haptic module 153 can be controlled by user selection or setting by the controller. For example, the haptic module 153 may output different vibrations in a combining manner or a sequential manner.

Besides vibration, the haptic module 153 can generate various other tactile effects, including an effect by stimulation such as a pin arrangement vertically moving to contact skin, a spray force or suction force of air through a jet orifice or a suction opening, a touch to the skin, a contact of an electrode, electrostatic force, an effect by reproducing the sense of cold and warmth using an element that can absorb or generate heat, and the like.

The haptic module 153 can also be implemented to allow the user to feel a tactile effect through a muscle sensation such as the user's fingers or arm, as well as transferring the tactile effect through direct contact. Two or more haptic modules 153 may be provided according to the particular configuration of the mobile terminal 100.

An optical output module 154 can output a signal for indicating an event generation using light of a light source. Examples of events generated in the mobile terminal 100 may include message reception, call signal reception, a missed call, an alarm, a schedule notice, an email reception, information reception through an application, and the like.

A signal output by the optical output module 154 may be implemented in such a manner that the mobile terminal emits monochromatic light or light with a plurality of colors. The signal output may be terminated as the mobile terminal senses that a user has checked the generated event, for example.

The interface unit 160 serves as an interface for external devices to be connected with the mobile terminal 100. For example, the interface unit 160 can receive data transmitted from an external device, receive power to transfer to elements and components within the mobile terminal 100, or transmit internal data of the mobile terminal 100 to such external device. The interface unit 160 may include wired or wireless headset ports, external power supply ports, wired or wireless data ports, memory card ports, ports for connecting a device having an identification module, audio input/output (I/O) ports, video I/O ports, earphone ports, or the like.

The identification module may be a chip that stores various information for authenticating authority of using the mobile terminal 100 and may include a user identity module (UIM), a subscriber identity module (SIM), a universal subscriber identity module (USIM), and the like. In addition, the device having the identification module (also referred to herein as an “identifying device”) may take the form of a smart card. Accordingly, the identifying device can be connected with the terminal 100 via the interface unit 160.

When the mobile terminal 100 is connected with an external cradle, the interface unit 160 can serve as a passage to allow power from the cradle to be supplied to the mobile terminal 100 or may serve as a passage to allow various command signals input by the user from the cradle to be transferred to the mobile terminal there through. Various command signals or power input from the cradle may operate as signals for recognizing that the mobile terminal is properly mounted on the cradle.

The memory 170 can store programs to support operations of the controller 180 and store input/output data (for example, phonebook, messages, still images, videos, etc.). The memory 170 may store data related to various patterns of vibrations and audio which are output in response to touch inputs on the touch screen.

The memory 170 may include one or more types of storage mediums including a Flash memory, a hard disk, a solid state disk, a silicon disk, a multimedia card micro type, a card-type memory (e.g., SD or DX memory, etc), a Random Access Memory (RAM), a Static Random Access Memory (SRAM), a Read-Only Memory (ROM), an Electrically Erasable Programmable Read-Only Memory (EEPROM), a Programmable Read-Only memory (PROM), a magnetic memory, a magnetic disk, an optical disk, and the like. The mobile terminal 100 may also be operated in relation to a network storage device that performs the storage function of the memory 170 over a network, such as the Internet.

The controller 180 may typically control the general operations of the mobile terminal 100. For example, the controller 180 may set or release a lock state for restricting a user from inputting a control command with respect to applications when a status of the mobile terminal meets a preset condition.

The controller 180 can also perform the controlling and processing associated with voice calls, data communications, video calls, and the like, or perform pattern recognition processing to recognize a handwriting input or a picture drawing input performed on the touch screen as characters or images, respectively. In addition, the controller 180 can control one or a combination of those components in order to implement various exemplary embodiments disclosed herein.

The power supply unit 190 receives external power or provide internal power and supply the appropriate power required for operating respective elements and components included in the mobile terminal 100. The power supply unit 190 may include a battery, which is typically rechargeable or be detachably coupled to the terminal body for charging.

The power supply unit 190 may include a connection port. The connection port may be configured as one example of the interface unit 160 to which an external charger for supplying power to recharge the battery is electrically connected.

As another example, the power supply unit 190 may be configured to recharge the battery in a wireless manner without use of the connection port. In this example, the power supply unit 190 can receive power, transferred from an external wireless power transmitter, using at least one of an inductive coupling method which is based on magnetic induction or a magnetic resonance coupling method which is based on electromagnetic resonance.

Various embodiments described herein may be implemented in a computer-readable medium, a machine-readable medium, or similar medium using, for example, software, hardware, or any combination thereof.

Referring now to FIGS. 1B and 1C, the mobile terminal 100 is described with reference to a bar-type terminal body. However, the mobile terminal 100 may alternatively be implemented in any of a variety of different configurations. Examples of such configurations include watch-type, clip-type, glasses-type, or as a folder-type, flip-type, slide-type, swing-type, and swivel-type in which two and more bodies are combined with each other in a relatively movable manner, and combinations thereof. Discussion herein will often relate to a particular type of mobile terminal (for example, bar-type, watch-type, glasses-type, and the like). However, such teachings with regard to a particular type of mobile terminal will generally apply to other types of mobile terminals as well.

The mobile terminal 100 will generally include a case (for example, frame, housing, cover, and the like) forming the appearance of the terminal. In this embodiment, the case is formed using a front case 101 and a rear case 102. Various electronic components are incorporated into a space formed between the front case 101 and the rear case 102. At least one middle case may be additionally positioned between the front case 101 and the rear case 102.

The display unit 151 is shown located on the front side of the terminal body to output information. As illustrated, a window 151a of the display unit 151 may be mounted to the front case 101 to form the front surface of the terminal body together with the front case 101.

In some embodiments, electronic components may also be mounted to the rear case 102. Examples of such electronic components include a detachable battery 191, an identification module, a memory card, and the like. Rear cover 103 is shown covering the electronic components, and this cover may be detachably coupled to the rear case 102. Therefore, when the rear cover 103 is detached from the rear case 102, the electronic components mounted to the rear case 102 are externally exposed.

As illustrated, when the rear cover 103 is coupled to the rear case 102, a side surface of the rear case 102 is partially exposed. In some cases, upon the coupling, the rear case 102 may also be completely shielded by the rear cover 103. In some embodiments, the rear cover 103 may include an opening for externally exposing a camera 121b or an audio output module 152b.

The cases 101, 102, 103 may be formed by injection-molding synthetic resin or may be formed of a metal, for example, stainless steel (STS), aluminum (Al), titanium (Ti), or the like.

As an alternative to the example in which the plurality of cases form an inner space for accommodating components, the mobile terminal 100 may be configured such that one case forms the inner space. In this example, a mobile terminal 100 having a uni-body is formed in such a manner that synthetic resin or metal extends from a side surface to a rear surface.

If desired, the mobile terminal 100 may include a waterproofing unit (not shown) for preventing introduction of water into the terminal body. For example, the waterproofing unit may include a waterproofing member which is located between the window 151 a and the front case 101, between the front case 101 and the rear case 102, or between the rear case 102 and the rear cover 103, to hermetically seal an inner space when those cases are coupled.

FIGS. 1B and 1C depict certain components as arranged on the mobile terminal. However, it is to be understood that alternative arrangements are possible and within the teachings of the instant disclosure. Some components may be omitted or rearranged. For example, the first manipulation unit 123a may be located on another surface of the terminal body, and the second audio output module 152b may be located on the side surface of the terminal body.

The display unit 151 is generally configured to output information processed in the mobile terminal 100. For example, the display unit 151 may display execution screen information of an application program executing at the mobile terminal 100 or user interface (UI) and graphic user interface (GUI) information in response to the execution screen information.

The display unit 151 outputs information processed in the mobile terminal 100. The display unit 151 may be implemented using one or more suitable display devices. Examples of such suitable display devices include a liquid crystal display (LCD), a thin film transistor-liquid crystal display (TFT-LCD), an organic light emitting diode (OLED), a flexible display, a 3-dimensional (3D) display, an e-ink display, and combinations thereof.

The display unit 151 may be implemented using two display devices, which can implement the same or different display technology. For instance, a plurality of the display units 151 may be arranged on one side, either spaced apart from each other, or these devices may be integrated, or these devices may be arranged on different surfaces.

The display unit 151 may also include a touch sensor which senses a touch input received at the display unit. When a touch is input to the display unit 151, the touch sensor may be configured to sense this touch and the controller 180, for example, may generate a control command or other signal corresponding to the touch. The content which is input in the touching manner may be a text or numerical value, or a menu item which can be indicated or designated in various modes.

The touch sensor may be configured in a form of a film having a touch pattern, disposed between the window 151a and a display on a rear surface of the window 151 a, or a metal wire which is patterned directly on the rear surface of the window 151 a. Alternatively, the touch sensor may be integrally formed with the display. For example, the touch sensor may be disposed on a substrate of the display or within the display.

The display unit 151 may also form a touch screen together with the touch sensor. Here, the touch screen may serve as the user input unit 123 (see FIG. 1A). Therefore, the touch screen may replace at least some of the functions of the first manipulation unit 123a.

The first audio output module 152a may be implemented in the form of a speaker to output voice audio, alarm sounds, multimedia audio reproduction, and the like.

The window 151a of the display unit 151 will typically include an aperture to permit audio generated by the first audio output module 152a to pass. One alternative is to allow audio to be released along an assembly gap between the structural bodies (for example, a gap between the window 151a and the front case 101). In this case, a hole independently formed to output audio sounds may not be seen or is otherwise hidden in terms of appearance, thereby further simplifying the appearance and manufacturing of the mobile terminal 100.

The optical output module 154 can be configured to output light for indicating an event generation. Examples of such events include a message reception, a call signal reception, a missed call, an alarm, a schedule notice, an email reception, information reception through an application, and the like. When a user has checked a generated event, the controller can control the optical output unit 154 to stop the light output.

The first camera 121a can process image frames such as still or moving images obtained by the image sensor in a capture mode or a video call mode. The processed image frames can then be displayed on the display unit 151 or stored in the memory 170.

The first and second manipulation units 123a and 123b are examples of the user input unit 123, which may be manipulated by a user to provide input to the mobile terminal 100. The first and second manipulation units 123a and 123b may also be commonly referred to as a manipulating portion, and may employ any tactile method that allows the user to perform manipulation such as touch, push, scroll, or the like. The first and second manipulation units 123a and 123b may also employ any non-tactile method that allows the user to perform manipulation such as proximity touch, hovering, or the like.

FIG. 1B illustrates the first manipulation unit 123a as a touch key, but possible alternatives include a mechanical key, a push key, a touch key, and combinations thereof.

Input received at the first and second manipulation units 123a and 123b may be used in various ways. For example, the first manipulation unit 123a may be used by the user to provide an input to a menu, home key, cancel, search, or the like, and the second manipulation unit 123b may be used by the user to provide an input to control a volume level being output from the first or second audio output modules 152a or 152b, to switch to a touch recognition mode of the display unit 151, or the like.

As another example of the user input unit 123, a rear input unit (not shown) may be located on the rear surface of the terminal body. The rear input unit can be manipulated by a user to provide input to the mobile terminal 100. The input may be used in a variety of different ways. For example, the rear input unit may be used by the user to provide an input for power on/off, start, end, scroll, control volume level being output from the first or second audio output modules 152a or 152b, switch to a touch recognition mode of the display unit 151, and the like. The rear input unit may be configured to permit touch input, a push input, or combinations thereof.

The rear input unit may be located to overlap the display unit 151 of the front side in a thickness direction of the terminal body. As one example, the rear input unit may be located on an upper end portion of the rear side of the terminal body such that a user can easily manipulate it using a forefinger when the user grabs the terminal body with one hand. Alternatively, the rear input unit can be positioned at most any location of the rear side of the terminal body.

Embodiments that include the rear input unit may implement some or all of the functionality of the first manipulation unit 123a in the rear input unit. As such, in situations where the first manipulation unit 123a is omitted from the front side, the display unit 151 can have a larger screen.

As a further alternative, the mobile terminal 100 may include a finger scan sensor which scans a user's fingerprint. The controller 180 can then use fingerprint information sensed by the finger scan sensor as part of an authentication procedure. The finger scan sensor may also be installed in the display unit 151 or implemented in the user input unit 123.

The microphone 122 is shown located at an end of the mobile terminal 100, but other locations are possible. If desired, multiple microphones may be implemented, with such an arrangement permitting the receiving of stereo sounds.

The interface unit 160 may serve as a path allowing the mobile terminal 100 to interface with external devices. For example, the interface unit 160 may include one or more of a connection terminal for connecting to another device (for example, an earphone, an external speaker, or the like), a port for near field communication (for example, an Infrared Data Association (IrDA) port, a Bluetooth port, a wireless LAN port, and the like), or a power supply terminal for supplying power to the mobile terminal 100. The interface unit 160 may be implemented in the form of a socket for accommodating an external card, such as Subscriber Identification Module (SIM), User Identity Module (UIM), or a memory card for information storage.

The second camera 121b is shown located at the rear side of the terminal body and includes an image capturing direction that is substantially opposite to the image capturing direction of the first camera unit 121a. If desired, second camera 121a may alternatively be located at other locations, or made to be moveable, in order to have a different image capturing direction from that which is shown.

The second camera 121b can include a plurality of lenses arranged along at least one line. The plurality of lenses may also be arranged in a matrix configuration. The cameras may be referred to as an “array camera.”

When the second camera 121b is implemented as an array camera, images may be captured in various manners using the plurality of lenses and images with better qualities.

As shown in FIG. 1C, a flash 124 is shown adjacent to the second camera 121b. When an image of a subject is captured with the camera 121b, the flash 124 may illuminate the subject.

As shown in FIG. 1B, the second audio output module 152b can be located on the terminal body. The second audio output module 152b may implement stereophonic sound functions in conjunction with the first audio output module 152a, and may be also used for implementing a speaker phone mode for call communication.

At least one antenna for wireless communication may be located on the terminal body. The antenna may be installed in the terminal body or formed by the case. For example, an antenna which configures a part of the broadcast receiving module 111 may be retractable into the terminal body. Alternatively, an antenna may be formed using a film attached to an inner surface of the rear cover 103, or a case that includes a conductive material.

A power supply unit 190 for supplying power to the mobile terminal 100 may include a battery 191, which is mounted in the terminal body or detachably coupled to an outside of the terminal body. The battery 191 may receive power via a power source cable connected to the interface unit 160. Also, the battery 191 can be recharged in a wireless manner using a wireless charger. Wireless charging may be implemented by magnetic induction or electromagnetic resonance.

The rear cover 103 is shown coupled to the rear case 102 for shielding the battery 191, to prevent separation of the battery 191, and to protect the battery 191 from an external impact or from foreign material. When the battery 191 is detachable from the terminal body, the rear case 103 may be detachably coupled to the rear case 102.

An accessory for protecting an appearance or assisting or extending the functions of the mobile terminal 100 can also be provided on the mobile terminal 100. As one example of an accessory, a cover or pouch for covering or accommodating at least one surface of the mobile terminal 100 may be provided. The cover or pouch may cooperate with the display unit 151 to extend the function of the mobile terminal 100. Another example of the accessory is a touch pen for assisting or extending a touch input to a touch screen.

The mobile terminal of the present invention is configured to collect a bio-information of a user by contacting part of the user's body. The mobile terminal according to an embodiment of the present invention measures a blood pressure using at least one bio-information. Hereinafter, a detailed structure of the mobile terminal for measuring a blood pressure will be explained.

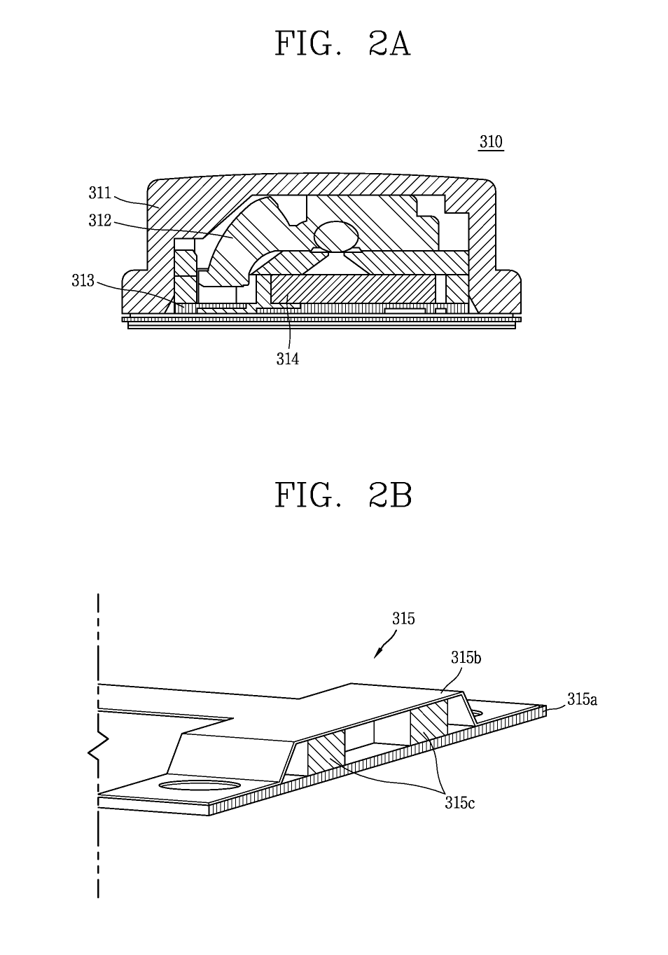

FIG. 2A is a sectional view illustrating a structure of a sensing module capable of collecting bio-information. FIG. 2B is a conceptual view illustrating a pressure sensor. Referring to FIGS. 1B and 2A, the sensing module 310 may be integrally formed with a signal input unit for generating a control command. The mobile terminal according to an embodiment of the present invention measures a blood pressure, based on information on a pulse wave sensed while an external force is being received. That is, the mobile terminal may calculate a blood pressure based on information on a sensed pressure and a sensed pulse wave.

For instance, the sensing module 310 may be formed on a front surface of the mobile terminal 100, and may be configured as a button for generating a control signal when pressed. Although not shown, the sensing module 310 may include an actuator configured to generate a different voltage when pressed, etc.

The sensing module 310 includes a cover portion 311, a lens portion 312, a light emitting portion 313, a light receiving portion 314, and a pressure sensor 315. The cover portion 311 forms the appearance of the sensing module 310, and contacts part of a user's body. The cover portion 311 is configured to accommodate therein the lens portion 312, the light emitting portion 313, the light receiving portion 314 and the pressure sensor 315, and is configured to be moved in a thickness direction of the mobile terminal 100 by an external force. The cover portion 311 may include an elastic member moved by an external force and restored to the original state.

The cover portion 311 may include an open region or a transmissive region formed to transmit light emitted from the light emitting portion 313 to a human body. The light emitting portion 313 may be configured as an IR light emitting diode (LED). The lens portion 312 is disposed on the light emitting portion 313. The lens portion 312 is configured to reflect light such that the light emitted from the light emitting portion 313 is concentrated to one region of the cover portion 311. The one region may be an open region or a transmissive region of the cover portion 311. The lens portion 312 may be formed such that light emitted from the light emitting portion 313 and reflected from a human body disposed on the cover portion 311 may be transmitted to the light receiving portion 314.

The light receiving portion 314 is configured to sense light of an IR LED reflected from a finger. The light receiving portion 314 may have an array structure. In this case, the light receiving portion 314 may sense a motion of the finger on the cover portion 311, based on light reflected from the finger.

The pressure sensor 315 is disposed in the cover portion 311 so as to sense an external force applied to the cover portion 311. The pressure sensor 315 may sense an external force applied from the outside, by supporting at least one of the cover portion 311, the lens portion 312, the light emitting portion 313 and the light receiving portion 314.

Referring to FIG. 2B, the pressure sensor 315 may include a supporting portion 315a, a sensing portion 315b, and an elastic portion 315c disposed between the sensing portion 315b and the supporting portion 315a. The pressure sensor 315 is formed to be transformable by an external force, and is configured to sense a pressure based on a voltage value changed due to its transformation.

The sensing module 310 may further include a leaf spring for supporting the cover portion 311 to which an external force is applied. And the pressure sensor 315 is disposed to face the leaf spring, and may include a pressurization structure pressed by an external force.

In the mobile terminal according to an embodiment of the present invention, a signal input unit for inputting a control signal by having a pressurization structure includes a PPG sensor and a pressure sensor. Thus, information for calculating a blood pressure may be obtained without an additional space for the PPG sensor and the pressure sensor. Further, since the PPG sensor and the pressure sensor are integrally formed with the signal input unit, bio information for calculating a blood pressure may be obtained while a specific function is being executed.

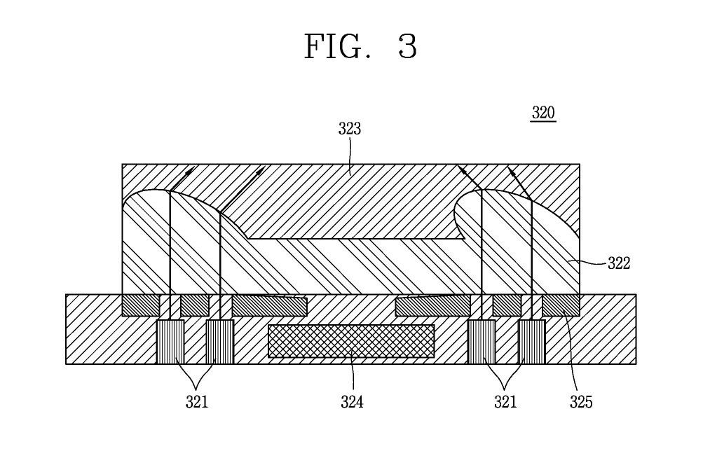

FIG. 3 is a conceptual view illustrating a sensing module according to an embodiment of the present invention. The mobile terminal according to an embodiment of the present invention calculates a blood pressure using information on a pressure and a pulse wave.

Referring to FIG. 3, a sensing module 320 is configured to sense a pulse wave and a fingerprint, and the controller 180 measures a blood pressure using a sensed pulse wave and a sensed pressure. The sensing module 320 may include a plurality of light emitting portions 321, a lens portion 322, a cover portion 323, a light receiving portion 324 and a matrix layer 325. The light emitting portions 321 may be configured as light emitting diodes (LED).

Light emitted from the light emitting portions 321 and passing through the matrix layer 325 is reflected from a finger disposed close to the cover portion 323, through the lens portion 322.

The sensing module 320 according to an embodiment of the present invention may sense a motion of a finger by the plurality of light emitting portions 321 and the matrix layer 325. The controller 180 may generate a control command based on the motion of the finger.

The controller 180 may sense a pulse wave based on light reflected from a finger which has contacted the sensing module 320, using the plurality of light emitting portions 321 and the light receiving portion 324. Although not shown, the sensing module 320 includes a pressure sensor. The pressure sensor may be the same pressure sensor shown in FIG. 2B. The controller 180 may measure a blood pressure based on a measured pulse wave and a measured pressure.

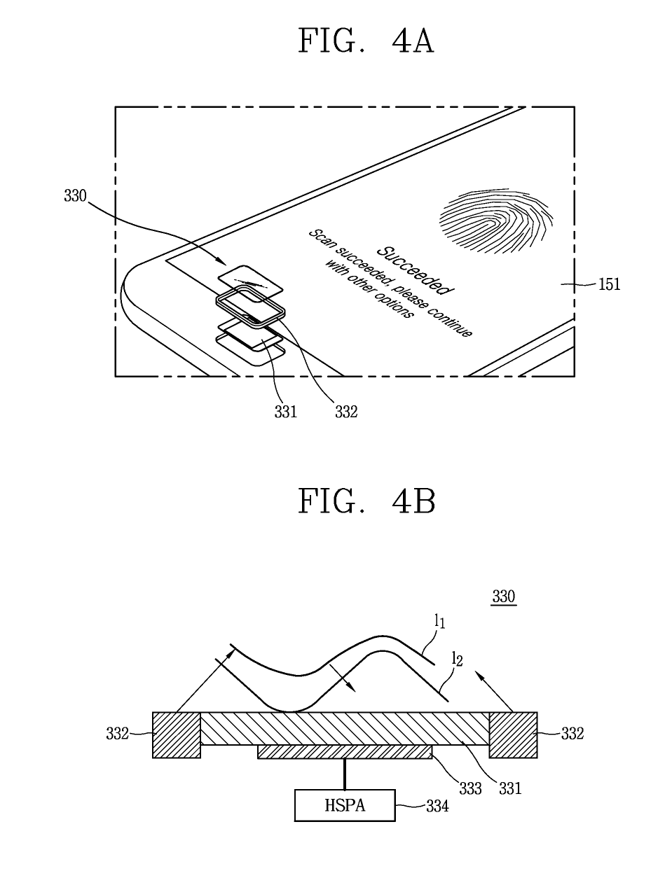

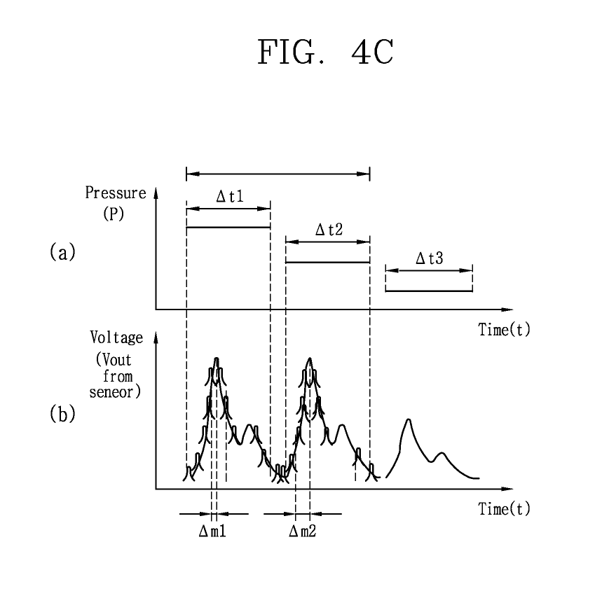

FIGS. 4A and 4B are conceptual views illustrating a sensing module capable of sensing a fingerprint according to an embodiment of the present invention. The sensing module for sensing a fingerprint and a fingerprint change according to an embodiment of the present invention serves as an electrode unit of an ECG module for measuring an electrocardiogram.

Referring to FIGS. 4A and 4B, a sensing module 330 according to this embodiment includes a first region 331 and a second region 332. The first region 331 is formed of an Rx electrode, and the second region 332 is formed of a Tx electrode. The first region 331 may be formed to have a predetermined width such that a user's hand may contact, and the second region 332 may be formed to enclose an edge part of the first region 331. The first and second regions 331, 332 are exposed to the outside of the mobile terminal 100, and may have any shape.

The sensing module 330 may sense a fingerprint in an active capacitive manner by using the first and second regions 331, 332. An electric signal is transmitted from the second region 332 formed of a metallic material, and the transmitted electric signal is reflected by a curved shape of a fingerprint. The transmitted electric signal passes through a dead skin 12 of a finger, and is reflected from a live skin 11 to thus be incident onto the second region 332. The first region 331 formed of a pixel array 333 disposed below the second region 332 senses a difference of capacitance of an electrical signal, thereby forming a fingerprint image. The sensing module 330 may further include a High Sensitive Pixel Amplifier (HSPA) 334.

The second region 332 may be formed of a first electrode between a pair of electrodes which contact a human body in order to measure an electrocardiogram based on a potential difference. The controller 180 may measure an electrocardiogram based on a potential difference occurring when the pair of electrodes contact different regions of a human body, by using the second region 332 as a first electrode, and by using a metallic ember mounted to another region of the mobile terminal 100 as a second electrode.

The electrocardiogram (ECG) is a process of recording an electrical activity of the heart over a period of time using electrodes placed on a patient's body. These electrodes detect the tiny electrical changes on the skin that arise from the heart muscle depolarizing during each heartbeat. The ECG corresponds to information for diagnosing a motion of the heart.

That is, the mobile terminal according to this embodiment may calculate a blood pressure based on an electrocardiogram, and based on a pulse wave collected by a PPG sensor. In this case, the electrocardiogram is collected by a first electrode implemented as the second region 332 formed of a metallic material and included in the sensing module 330 having a fingerprint sensing function, and a second electrode disposed on another region of the mobile terminal.

As aforementioned, the mobile terminal includes an ECG sensor including electrodes which contact different regions of a human body in order to measure a blood pressure, and a PPG sensor including a light emitting portion and a light receiving portion so as to collect a pulse wave. Hereinafter, will be explained the sensing module disposed on one region of the mobile terminal.

FIG. 4C is a view illustrating a pulse wave measured while a pressure is applied.

FIG. 4C(a) illustrates a pressure applied as time lapses. Referring to FIG. 4C(b), a pulse wave sensed by the PPG sensor is shown as a waveform indicating a voltage change by lapse of time.

The light emitting portions 313 and the light receiving portion 314 constitute the PPG sensor. The PPG sensor irradiates light of a specific wavelength to a human body, and measures a pulsation component resulting from a heartbeat based on reflected or transmitted light. Once light is irradiated to a human body from the light emitting portions 310, the light is absorbed by blood, bones and tissues. Part of the absorbed light reaches the light receiving portion 314. An absorbed degree of the light is proportional to an amount of the skin, tissues and blood, and is changed only when a blood flow is changed by a heartbeat. Thus, a change in an absorbed optical amount is proportional to a blood change. An amount of light received by the light receiving portion 314 is obtained by deducting an amount of light absorbed by a finger, from a total amount of light which has penetrated the light receiving portion 314. Thus, a change of an optical amount of transmitted light also indicates a change of a blood flow. Accordingly, it is possible to detect a change of a blood amount by a heartbeat, by measuring an amount of light received by the light receiving portion 314.

The controller 180 estimates blood pressures of a region to be tested, based on differences between time points corresponding to peaks of a sensed pulse wave, and time points corresponding to peaks of a filtered pulse wave. Among the estimated blood pressures, a maximum blood pressure may be defined as a systolic BP, and a minimum blood pressure may be defined as diastolic BP. Other blood pressures such as a mean BP may be estimated based on the estimated blood pressures.

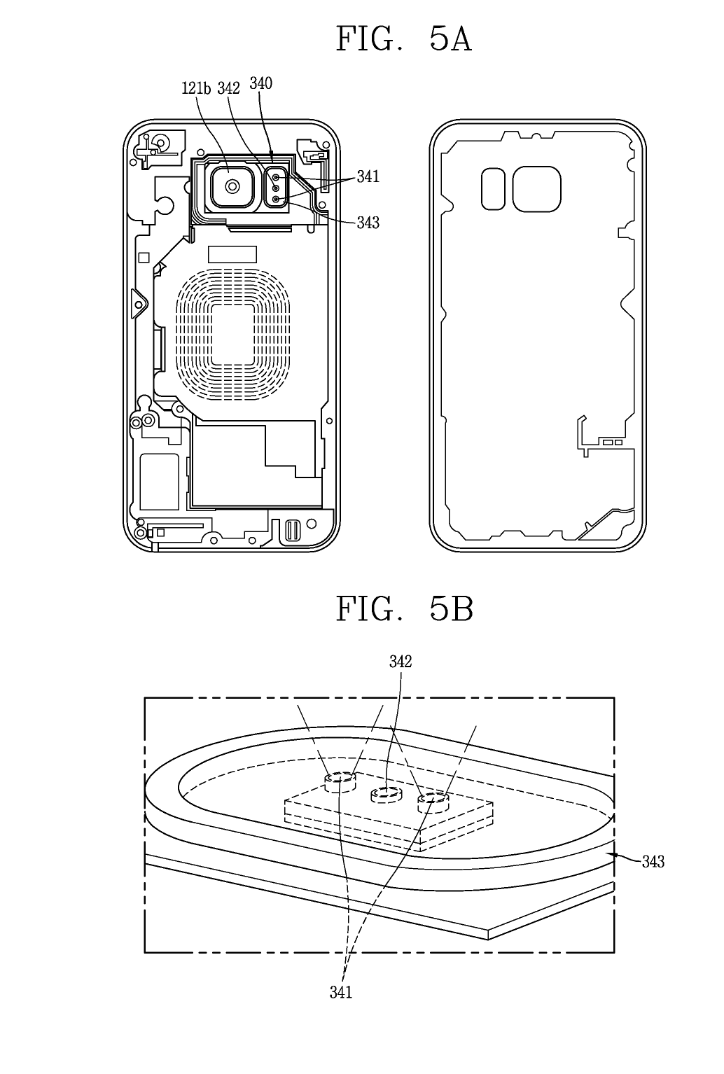

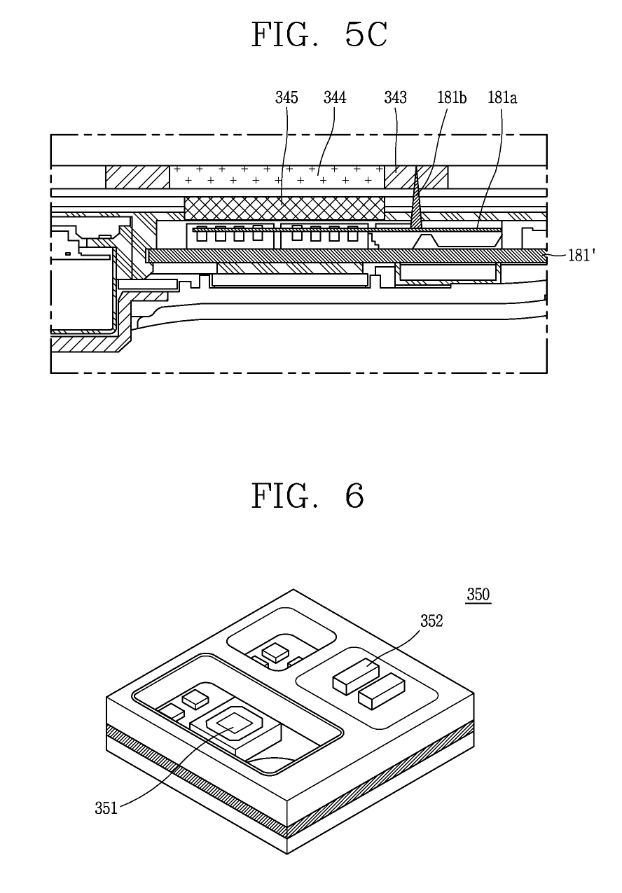

FIGS. 5A to 5C are conceptual views illustrating a sensing module according to another embodiment of the present invention.

The sensing module according to this embodiment includes a PPG sensor for collecting pulse wave information by measuring a distance of a subject, and includes part of an ECG module for measuring an electrocardiogram, the part formed as an additional metallic member. Thus, the mobile terminal may calculate a blood pressure based on pulse wave information and electrocardiogram information.

Referring to FIGS. 5A and 5B, the mobile terminal according to this embodiment of the present invention measures a blood pressure based on a pulse wave and an electrocardiogram obtained by a sensing module 340 and an additional electrode. One region of the sensing module 340 is formed to be exposed to a rear surface of the mobile terminal 100.

Preferably, the sensing module 340 is disposed close to the rear camera 121b. The sensing module 340 includes a plurality of light emitting portions 341, a light receiving portion 342, and an electrode unit 343. The flash 124 may be disposed close to the sensing module 340.

While the rear camera 121b is being activated, the emitting portions 341 and the light receiving portion 342 of the sensing module 340 may measure a distance between the mobile terminal 100 and a specific object. The controller 180 may execute an auto focusing for controlling a focal point of a subject obtained by the rear camera 121b, using the distance measuring function of the light emitting portions 341 and the light receiving portion 342.

For instance, the light emitting portions 341 may emit laser. Based on a difference between time when the laser has been emitted and time when the emitted laser is received by the light receiving portion 342 after being reflected from an arbitrary subject, a distance between the mobile terminal 100 and the subject may be measured. However, a type of light emitted from the light emitting portions 341 is not limited to laser.

The controller 180 may measure a pulse wave by the light emitting portions 341 and the light receiving portion 342. In a case where part of a user's body (e.g., finger) is close to the sensing module 340, if light emitted from the light emitting portions 341 and reflected from the user's body reaches the light receiving portion 342, the controller may measure a pulse wave using a pattern of light incident onto the light receiving portion 342.

Referring to FIG. 5C, the electrode unit 343 may be formed to enclose the light emitting portions 341 and the light receiving portion 342. The electrode unit 343 is mounted to be exposed to the outside of the mobile terminal 100. The sensing module 340 may further include a chip 345 electrically connected to the light emitting portions 341 and the light receiving portion 342, and a window 344 for covering the chip 345. The electrode unit 343 may be arranged to constitute an external surface of the mobile terminal, together with the window 344. And the electrode unit 343 may be formed on the same surface as the window 344.

The sensing module 340 is electrically connected to a main printed circuit board 181′ through a flexible printed circuit board 181a. The electrode unit 343 is connected to the flexible printed circuit board 181a through a connection portion 181b.

Preferably, the electrode unit 343 is disposed close to the light emitting portions 341 and the light receiving portion 342. With such a configuration, if a finger contacts the electrode potion 343, light emitted from the light emitting portions 341 is reflected from the finger to reach the light receiving portion 342.

If it is determined based on a pattern of light incident onto the light receiving portion 342 that a user's body has contacted the electrode unit 343, the controller 180 controls the electrode unit 343 to apply a current, and collects information on a blood pressure and a pulse wave.

In this embodiment, an additional electrode unit which contacts part of a user's body may be disposed on one region of the mobile terminal such that a potential difference between the additional electrode unit and the electrode unit 343 may occur. For instance, the additional electrode unit may be formed at a metallic member which forms an edge of a signal input module disposed on a front surface of the mobile terminal, or a metallic member which encloses the audio output module 152.

If a potential difference occurs as a user's body contact a pair of electrode portions, the controller 180 may control the sensing module 340 to collect information on a pulse wave by the light emitting portions 341 and the light receiving portion 342.

The sensing module 340 may be used to execute an auto focusing or to measure a pulse wave of a human body. The controller 180 may selectively execute a function based on a specific control command applied by a user, or may activate a function to collect user's bio information when information of a specific pattern is collected by the electrode unit or the light receiving portion. Since an additional configuration for collecting bio information is not required, the mobile terminal may have a simplified structure.

The sensing module 340 may not include the electrode unit 343. That is, the sensing module 340 may be formed only to collect information on a pulse wave of a human body, without including the electrode unit 343 which constitutes an ECG module. In this case, a plurality of electrode portions which constitutes the ECG module may be formed on another region of the mobile terminal 100.

FIG. 6 is a conceptual view illustrating a sensing module 350 according to still another embodiment of the present invention. The sensing module 350 according to this embodiment includes a plurality of sensors for sensing different objects. The sensing module 350 is provided with a housing having an inner space, and the plurality of sensors are mounted to the housing.

The sensing module 350 includes a PPG module 351 for measuring a pulse wave, and an electrode unit 352 for measuring an electrocardiogram. Although not shown, the PPG module 351 includes a light emitting portion and a light receiving portion. The sensing module 350 may further include an oxygen saturation sensor, a body temperature sensor, etc.

The sensing module 350 may be disposed on a rear surface of the mobile terminal. However, the present invention is not limited to this. Preferably, the PPG module 351 and the electrode unit 352 are disposed to be exposed to the outside. The electrode unit 352 constitutes an ECC module for measuring a potential difference of a current applied to a human body, together an additional electrode unit disposed on another region of the mobile terminal.

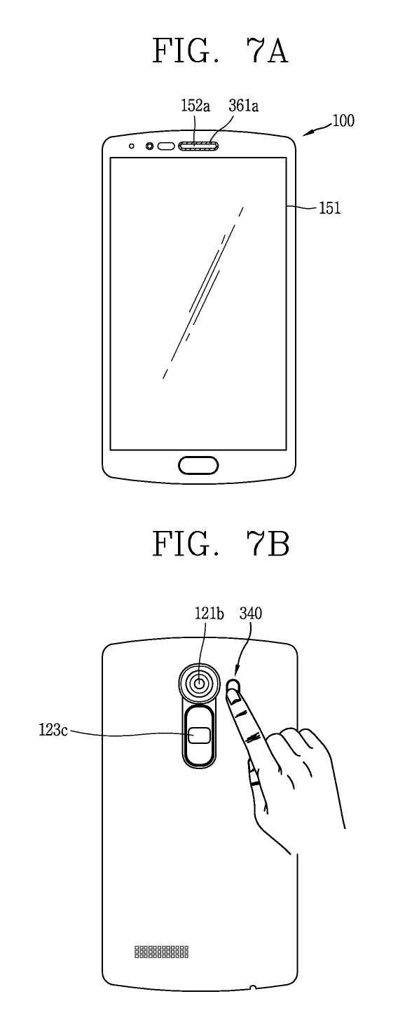

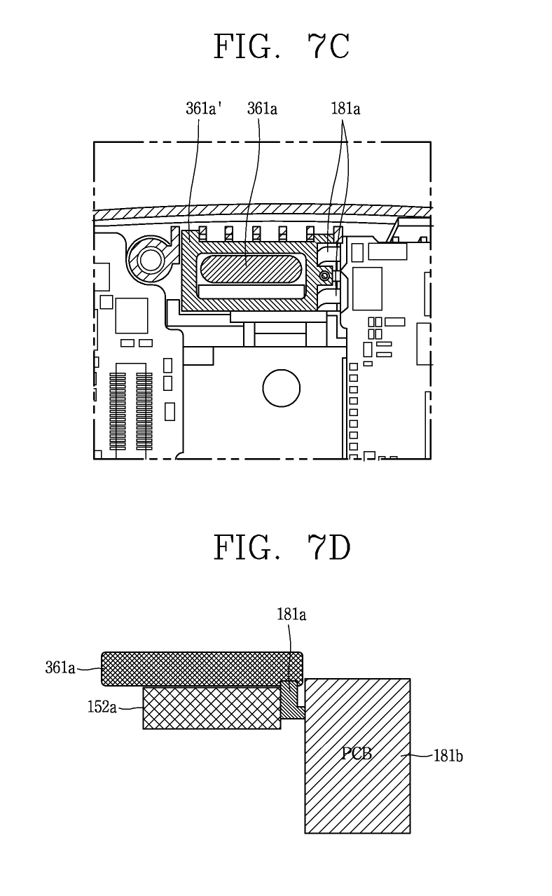

FIGS. 7A and 7B are conceptual views illustrating an ECG module according to an embodiment of the present invention, and FIGS. 7C and 7D are conceptual views illustrating an electrode unit disposed close to an audio output module 152a.

The ECG module is composed of a pair of electrode portions which contact different regions of a human body. Alternatively, the mobile terminal 100 may include one electrode unit, and an external device wirelessly connected to the mobile terminal 100 may include another electrode unit.

Referring to FIGS. 7A and 7B, a first electrode unit 361a which constitutes the ECG module according to this embodiment is disposed close to the audio output unit module 152a, and a second electrode unit 361b is disposed close to the sensing module 140. The sensing module 140 includes a light emitting portion for emitting laser, and a light receiving portion for collecting laser reflected from an arbitrary subject. The controller 180 may focalize the rear camera 121b, using incident light.

The sensing module 140 and the second electrode unit 361b are the same as the sensing module 340 shown in FIGS. 5A to 5C. The second electrode unit 361b may be implemented as a metallic member which encloses the sensing module 140, and is exposed to the outside of the mobile terminal. The controller 180 may collect information on a pulse wave based on light reflected from a human body close to the sensing module 140, by the light emitting portion and the light receiving portion of the sensing module 140. That is, the sensing module 140 may serve as a PPG sensor.

Referring to FIGS. 7C and 7D, the first electrode unit 361a may serve as a cover for covering a speaker module which implements the audio output unit 152a. That is, the first electrode unit 361a is exposed to the outside with covering the speaker module. The first electrode unit 361a may be formed of metal mesh such that a sound (vibrations) generated from the speaker module may be transmitted to the outside.

A metallic extended portion 361a′ may be formed on a contact terminal 181a, and the first electrode unit 361a exposed to the outside may be disposed on the metallic extended portion 361a′. The contact terminal 181a is electrically connected to a printed circuit board 181b.

In this embodiment, if an ear of a user contacts the first electrode unit 361a and a hand of the user contacts the second electrode unit 361b, the controller 180 may measure an electrocardiogram based on a potential difference between the first and second electrode portions 361a, 361b. The controller 180 may measure a pulse wave using light (laser) reflected from the user's hand contacting the sensing module 140. The controller 180 may calculate a blood pressure based on the electrocardiogram and the pulse wave.

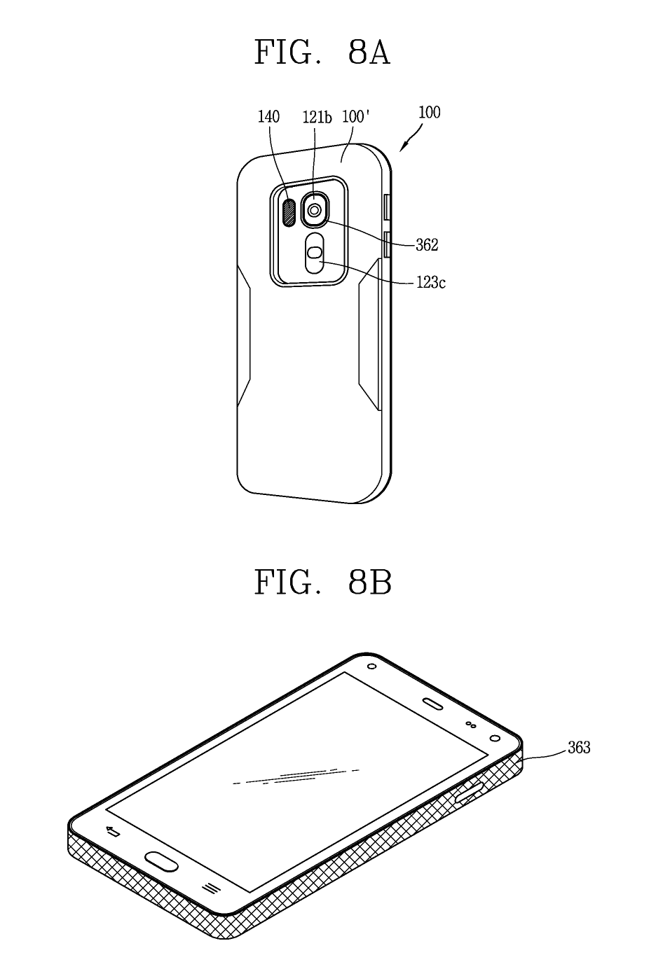

FIGS. 8A and 8B are conceptual views illustrating an electrode unit according to another embodiment of the present invention.

FIG. 8A illustrates the mobile terminal where a case 100′ is mounted to the terminal body. The case 100′ includes an open region through which part of the terminal body is exposed to the outside. The open region is formed to correspond to a component of the mobile terminal exposed to the outside (e.g., the signal input unit, the camera, the sensing module, etc.) when the case 100′ is mounted to the terminal body.

The sensing module 140, the rear camera 121b and the rear key 123c of the mobile terminal 100 are exposed to the outside based on the open region. One of the pair of electrode portions which constitute the ECG module is disposed on one region of the terminal body which is exposed to the outside through the open region. For instance, an electrode unit 362 according to this embodiment may be formed along an edge of the rear camera 121b. The electrode unit 362 may be formed of a metallic member, metal deco to implement a sophisticated sense of the rear camera 121b.

Alternatively, the electrode unit 362 may be formed as a metallic member disposed close to the rear key 123c. In this case, while a user applies a control command using the rear key 123c, the controller 180 may collect bio information.

In this embodiment, even when a case for covering part of an external surface of the terminal body is mounted to the mobile terminal, the electrode unit may contact a user's body because it is exposed to the outside.

Referring to FIG. 8B, the electrode unit may be part of the terminal body. In a case where one region of the terminal body is formed of a metallic material, the one region may serve as an electrode unit 363. The electrode unit 363 may be formed to constitute a side surface of the terminal body. In this case, an electrocardiogram may be measured in a state where a user holds the mobile terminal, without a step of inputting an additional control command or touching a specific region by the user.

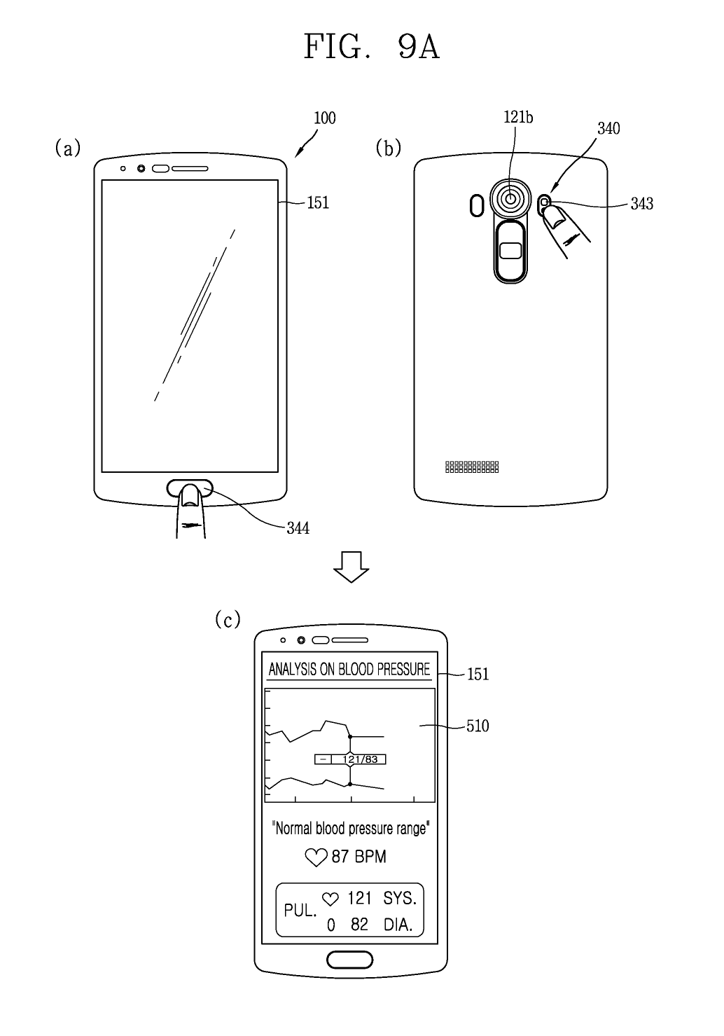

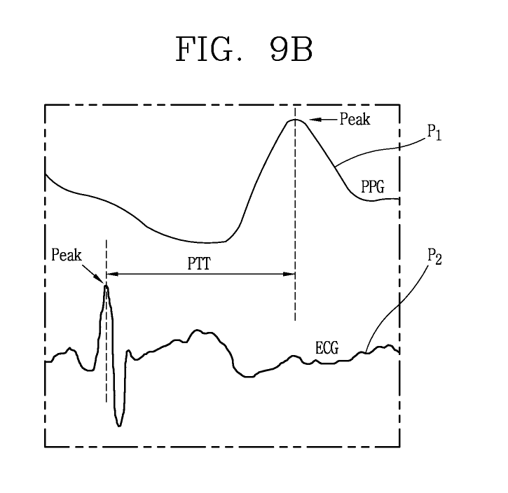

FIG. 9A is a conceptual view illustrating a control method of measuring a blood pressure, and FIG. 9B is a conceptual view illustrating a method of measuring a blood pressure based on information on an electrocardiogram (ECG) and a pulse wave.

The mobile terminal according to this embodiment measures a blood pressure in a cuff-less manner using information on an electrocardiogram (ECG) and a pulse wave.

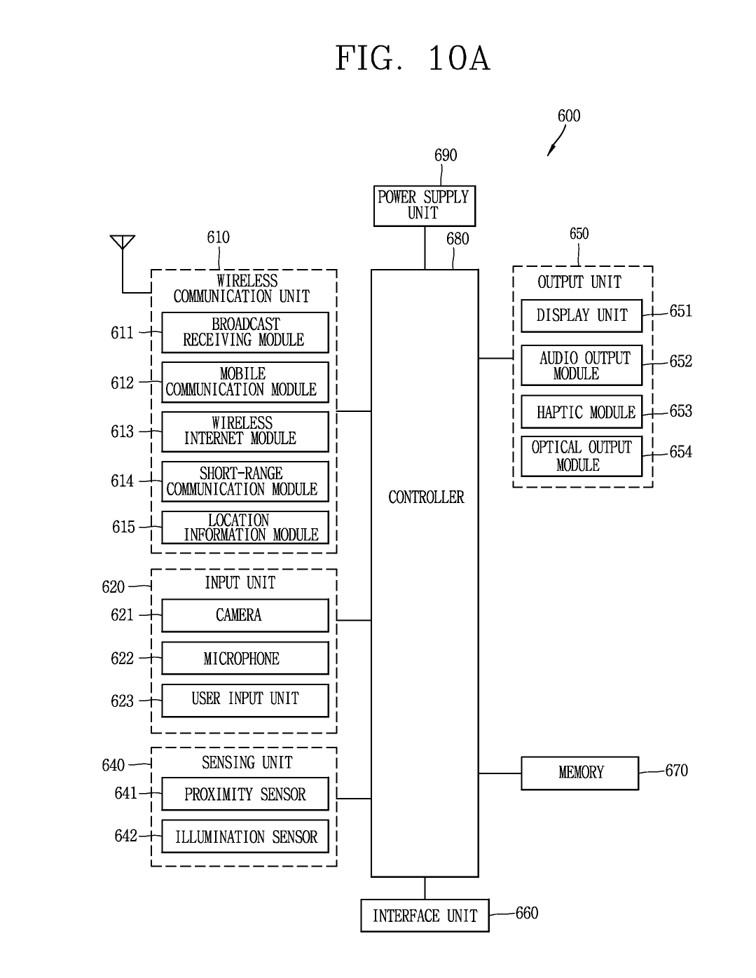

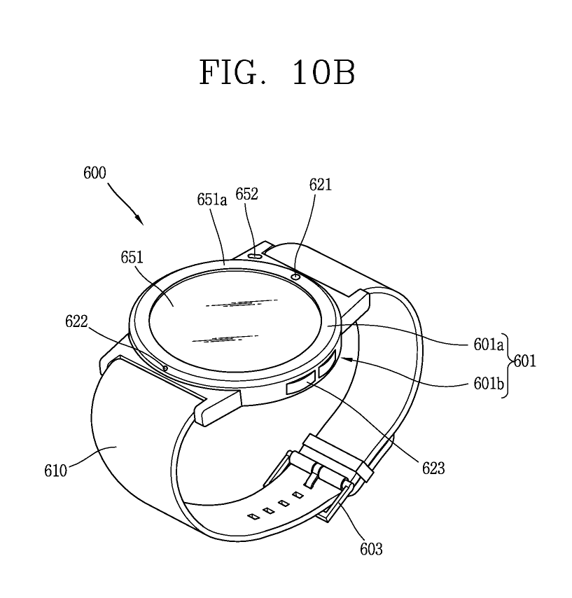

FIG. 9A(a) illustrates a front surface of the mobile terminal 100 including a first electrode unit 344, and FIG. 10A(b) illustrates a rear surface of the mobile terminal 100 including a sensing module 340 having a second electrode unit 343. The sensing module 340 is the same as the sensing module shown in FIG. 5A, and thus explanations about the sensing module 340 having the second electrode unit 343 will be replaced by those conducted with reference to FIGS. 5A to 5C. The first and second electrode portions 344, 343 constitute an ECG module for collecting electrocardiogram information.

Once the first and second electrode portions 344, 343 contact different parts of a user's body, the controller 180 measures an electrocardiogram based on a potential difference. Referring to FIG. 9B, the controller 180 calculates a blood pressure, based on a pulse wave pattern (P1) collected by a PPG sensor implemented as the sensing module 340, and based on an electrocardiogram pattern (P2) collected by an ECG module composed of the first and second electrode portions 344, 343. A pulse transit time (PPT) may be measured by using a time difference between a peak of the pulse wave pattern (P1) and a peak of the electrocardiogram pattern (P2). The controller 180 may calculate a blood pressure through a correlation between the PPT and a diastolic blood pressure.

The controller 180 may display the calculated blood pressure and related result information 510, on the display unit 151 of the mobile terminal 100.

A position of the first and second electrode portions 344, 343 according to this embodiment is not limited to the position shown in the drawings. The first and second electrode portions 344, 343 may be disposed on different regions of the mobile terminal which contact different parts of a human body.

FIG. 10A is a block diagram illustrating a mobile terminal according to another embodiment of the present invention. FIG. 10B is a conceptual view illustrating an example of a mobile terminal according to the present invention, which is viewed from one direction. The mobile terminal according to this embodiment corresponds to a watch-type mobile terminal wearable on a user's wrist.

The watch-type mobile terminal 600 is shown having components such as a wireless communication unit 610, an input unit 620, a sensing unit 640, an output unit 650, an interface unit 660, a memory 670, a controller 680, and a power supply unit 690. It is understood that implementing all of the illustrated components of FIG. 10A is not a requirement, and that greater or fewer components may alternatively be implemented.

Referring now to FIG. 10A, the watch-type mobile terminal 600 is shown having wireless communication unit 610 configured with several commonly implemented components. For instance, the wireless communication unit 610 typically includes one or more components which permit wireless communication between the watch-type mobile terminal 600 and a wireless communication system or network within which the mobile terminal is located.

The wireless communication unit 610 typically includes one or more modules which permit communications such as wireless communications between the watch-type mobile terminal 600 and a wireless communication system, communications between the watch-type mobile terminal 600 and another mobile terminal, communications between the watch-type mobile terminal 600 and an external server. Further, the wireless communication unit 610 typically includes one or more modules which connect the watch-type mobile terminal 600 to one or more networks. To facilitate such communications, the wireless communication unit 610 includes one or more of a broadcast receiving module 611, a mobile communication module 612, a wireless Internet module 613, a short-range communication module 614, and a location information module 615.

The input unit 620 includes a camera 621 for obtaining images or video, a microphone 622, which is one type of audio input device for inputting an audio signal, and a user input unit 623 (for example, a touch key, a push key, a mechanical key, a soft key, and the like) for allowing a user to input information. Data (for example, audio, video, image, and the like) is obtained by the input unit 620 and may be analyzed and processed by controller 680 according to device parameters, user commands, and combinations thereof.

The sensing unit 640 is typically implemented using one or more sensors configured to sense internal information of the mobile terminal, the surrounding environment of the mobile terminal, user information, and the like. For example, in FIG. 10A, the sensing unit 640 is shown having a proximity sensor 641 and an illumination sensor 642. If desired, the sensing unit 640 may alternatively or additionally include other types of sensors or devices, such as a touch sensor, an acceleration sensor, a magnetic sensor, a G-sensor, a gyroscope sensor, a motion sensor, an RGB sensor, an infrared (IR) sensor, a finger scan sensor, a ultrasonic sensor, an optical sensor (for example, camera 621), a microphone 622, a battery gauge, an environment sensor (for example, a barometer, a hygrometer, a thermometer, a radiation detection sensor, a thermal sensor, and a gas sensor, among others), and a chemical sensor (for example, an electronic nose, a health care sensor, a biometric sensor, and the like), to name a few. The watch-type mobile terminal 600 may be configured to utilize information obtained from sensing unit 640, and in particular, information obtained from one or more sensors of the sensing unit 640, and combinations thereof.

The output unit 650 is typically configured to output various types of information, such as audio, video, tactile output, and the like. The output unit 650 is shown having a display unit 651, an audio output module 652, a haptic module 653, and an optical output module 654.

The display unit 651 may have an inter-layered structure or an integrated structure with a touch sensor in order to facilitate a touch screen. The touch screen may provide an output interface between the watch-type mobile terminal 600 and a user, as well as function as the user input unit 623 which provides an input interface between the watch-type mobile terminal 600 and the user.

The interface unit 660 serves as an interface with various types of external devices that can be coupled to the watch-type mobile terminal 600. The interface unit 660, for example, may include any of wired or wireless ports, external power supply ports, wired or wireless data ports, memory card ports, ports for connecting a device having an identification module, audio input/output (I/O) ports, video I/O ports, earphone ports, and the like. In some cases, the watch-type mobile terminal 600 may perform assorted control functions associated with a connected external device, in response to the external device being connected to the interface unit 660.

The memory 670 is typically implemented to store data to support various functions or features of the watch-type mobile terminal 600. For instance, the memory 670 may be configured to store application programs executed in the watch-type mobile terminal 600, data or instructions for operations of the watch-type mobile terminal 600, and the like. Some of these application programs may be downloaded from an external server via wireless communication. Other application programs may be installed within the watch-type mobile terminal 600 at time of manufacturing or shipping, which is typically the case for basic functions of the watch-type mobile terminal 600 (for example, receiving a call, placing a call, receiving a message, sending a message, and the like). It is common for application programs to be stored in the memory 670, installed in the watch-type mobile terminal 600, and executed by the controller 680 to perform an operation (or function) for the watch-type mobile terminal 600.

The controller 680 typically functions to control overall operation of the watch-type mobile terminal 600, in addition to the operations associated with the application programs. The controller 680 may provide or process information or functions appropriate for a user by processing signals, data, information and the like, which are input or output by the various components depicted in FIG. 10A, or activating application programs stored in the memory 670. As one example, the controller 680 controls some or all of the components illustrated in FIGS. 10A and 10B according to the execution of an application program that have been stored in the memory 670.

The power supply unit 690 can be configured to receive external power or provide internal power in order to supply appropriate power required for operating elements and components included in the watch-type mobile terminal 600. The power supply unit 690 may include a battery, and the battery may be configured to be embedded in the terminal body, or configured to be detachable from the terminal body.

At least some of the above components may operate in a cooperating manner, so as to implement an operation or a control method of a glass type terminal according to various embodiments to be explained later. The operation or the control method of the glass type terminal may be implemented on the glass type terminal by driving at least one application program stored in the memory 670.

Referring still to FIG. 10A, various components depicted in this figure will now be described in more detail. Regarding the wireless communication unit 610, the broadcast receiving module 611 is typically configured to receive a broadcast signal and/or broadcast associated information from an external broadcast managing entity via a broadcast channel. The broadcast channel may include a satellite channel, a terrestrial channel, or both. In some embodiments, two or more broadcast receiving modules 611 may be utilized to facilitate simultaneously receiving of two or more broadcast channels, or to support switching among broadcast channels.

The mobile communication module 612 can transmit and/or receive wireless signals to and from one or more network entities. Typical examples of a network entity include a base station, an external mobile terminal, a server, and the like. Such network entities form part of a mobile communication network, which is constructed according to technical standards or communication methods for mobile communications (for example, Global System for Mobile Communication (GSM), Code Division Multi Access (CDMA), CDMA2000(Code Division Multi Access 2000), EV-DO(Enhanced Voice-Data Optimized or Enhanced Voice-Data Only), Wideband CDMA (WCDMA), High Speed Downlink Packet access (HSDPA), HSUPA(High Speed Uplink Packet Access), Long Term Evolution (LTE), LTE-A(Long Term Evolution-Advanced), and the like).

Examples of wireless signals transmitted and/or received via the mobile communication module 612 include audio call signals, video (telephony) call signals, or various formats of data to support communication of text and multimedia messages.

The wireless Internet module 613 is configured to facilitate wireless Internet access. This module may be internally or externally coupled to the watch-type mobile terminal 600. The wireless Internet module 613 may transmit and/or receive wireless signals via communication networks according to wireless Internet technologies.

Examples of such wireless Internet access include Wireless LAN (WLAN), Wireless Fidelity (Wi-Fi), Wi-Fi Direct, Digital Living Network Alliance (DLNA), Wireless Broadband (WiBro), Worldwide Interoperability for Microwave Access (WiMAX), High Speed Downlink Packet Access (HSDPA), Long Term Evolution (LTE), and the like. The wireless Internet module 613 may transmit/receive data according to one or more of such wireless Internet technologies, and other Internet technologies as well.

In some embodiments, when the wireless Internet access is implemented according to, for example, WiBro, HSDPA,HSUPA, GSM, CDMA, WCDMA, LTE, LTE-A and the like, as part of a mobile communication network, the wireless Internet module 613 performs such wireless Internet access. As such, the Internet module 613 may cooperate with, or function as, the mobile communication module 612.

The short-range communication module 614 is configured to facilitate short-range communications. Suitable technologies for implementing such short-range communications include BLUETOOTH™, Radio Frequency IDentification (RFID), Infrared Data Association (IrDA), Ultra-WideBand (UWB), ZigBee, Near Field Communication (NFC), Wireless-Fidelity (Wi-Fi), Wi-Fi Direct, Wireless USB(Wireless Universal Serial Bus), and the like. The short-range communication module 614 in general supports wireless communications between the watch-type mobile terminal 600 and a wireless communication system, communications between the watch-type mobile terminal 600 and another watch-type mobile terminal 600, or communications between the mobile terminal and a network where another watch-type mobile terminal 600 (or an external server) is located, via wireless area networks. One example of the wireless area networks is a wireless personal area networks.

In some embodiments, another mobile terminal (which may be configured similarly to watch-type mobile terminal 600) may be a wearable device, for example, a smart watch, a smart glass or a head mounted display (HMD), which is able to exchange data with the watch-type mobile terminal 600 (or otherwise cooperate with the watch-type mobile terminal 600). The short-range communication module 614 may sense or recognize the wearable device, and permit communication between the wearable device and the watch-type mobile terminal 600. In addition, when the sensed wearable device is a device which is authenticated to communicate with the watch-type mobile terminal 600, the controller 680, for example, may cause transmission of data processed in the watch-type mobile terminal 600 to the wearable device via the short-range communication module 614. Hence, a user of the wearable device may use the data processed in the watch-type mobile terminal 600 on the wearable device. For example, when a call is received in the watch-type mobile terminal 600, the user may answer the call using the wearable device. Also, when a message is received in the watch-type mobile terminal 600, the user can check the received message using the wearable device.

The location information module 615 is generally configured to detect, calculate, derive or otherwise identify a position of the mobile terminal. As an example, the location information module 615 includes a Global Position System (GPS) module, a Wi-Fi module, or both. If desired, the location information module 615 may alternatively or additionally function with any of the other modules of the wireless communication unit 610 to obtain data related to the position of the mobile terminal.

As one example, when the mobile terminal uses a GPS module, a position of the mobile terminal may be acquired using a signal sent from a GPS satellite. As another example, when the mobile terminal uses the Wi-Fi module, a position of the mobile terminal can be acquired based on information related to a wireless access point (AP) which transmits or receives a wireless signal to or from the Wi-Fi module.

The input unit 620 may be configured to permit various types of input to the mobile terminal 620. Examples of such input include audio, image, video, data, and user input. Image and video input is often obtained using one or more cameras 621. Such cameras 621 may process image frames of still pictures or video obtained by image sensors in a video or image capture mode. The processed image frames can be displayed on the display unit 651 or stored in memory 670. In some cases, the cameras 621 may be arranged in a matrix configuration to permit a plurality of images having various angles or focal points to be input to the watch-type mobile terminal 600. As another example, the cameras 621 may be located in a stereoscopic arrangement to acquire left and right images for implementing a stereoscopic image.

The microphone 622 is generally implemented to permit audio input to the watch-type mobile terminal 600. The audio input can be processed in various manners according to a function being executed in the watch-type mobile terminal 600. If desired, the microphone 622 may include assorted noise removing algorithms to remove unwanted noise generated in the course of receiving the external audio.

The user input unit 623 is a component that permits input by a user. Such user input may enable the controller 680 to control operation of the watch-type mobile terminal 600. The user input unit 623 may include one or more of a mechanical input element (for example, a key, a button located on a front and/or rear surface or a side surface of the watch-type mobile terminal 600, a dome switch, a jog wheel, a jog switch, and the like), or a touch-sensitive input, among others. As one example, the touch-sensitive input may be a virtual key or a soft key, which is displayed on a touch screen through software processing, or a touch key which is located on the mobile terminal at a location that is other than the touch screen. On the other hand, the virtual key or the visual key may be displayed on the touch screen in various shapes, for example, graphic, text, icon, video, or a combination thereof.

The user input unit 623 may recognize information sensed by the sensing unit 640, as well as by the aforementioned mechanical input means and touch type input means, as information input from a user. Accordingly, the controller 680 can control an operation of the watch-type mobile terminal 600 corresponding to the sensed information.

The sensing unit 640 is generally configured to sense one or more of internal information of the mobile terminal, surrounding environment information of the mobile terminal, user information, or the like. The controller 680 generally cooperates with the sending unit 640 to control operation of the watch-type mobile terminal 600 or execute data processing, a function or an operation associated with an application program installed in the mobile terminal based on the sensing provided by the sensing unit 640. The sensing unit 640 may be implemented using any of a variety of sensors, some of which will now be described in more detail.

The proximity sensor 641 may include a sensor to sense presence or absence of an object approaching a surface, or an object located near a surface, by using an electromagnetic field, infrared rays, or the like without a mechanical contact. The proximity sensor 641 may be arranged at an inner region of the mobile terminal covered by the touch screen, or near the touch screen.

The proximity sensor 641, for example, may include any of a transmissive type photoelectric sensor, a direct reflective type photoelectric sensor, a mirror reflective type photoelectric sensor, a high-frequency oscillation proximity sensor, a capacitance type proximity sensor, a magnetic type proximity sensor, an infrared rays proximity sensor, and the like. When the touch screen is implemented as a capacitance type, the proximity sensor 641 can sense proximity of a pointer relative to the touch screen by changes of an electromagnetic field, which is responsive to an approach of an object with conductivity. In this case, the touch screen (touch sensor) may also be categorized as a proximity sensor.

The term “proximity touch” will often be referred to herein to denote the scenario in which a pointer is positioned to be proximate to the touch screen without contacting the touch screen. The term “contact touch” will often be referred to herein to denote the scenario in which a pointer makes physical contact with the touch screen. For the position corresponding to the proximity touch of the pointer relative to the touch screen, such position will correspond to a position where the pointer is perpendicular to the touch screen. The proximity sensor 641 may sense proximity touch, and proximity touch patterns (for example, distance, direction, speed, time, position, moving status, and the like).

In general, controller 680 processes data corresponding to proximity touches and proximity touch patterns sensed by the proximity sensor 641, and cause output of visual information on the touch screen. In addition, the controller 680 can control the watch-type mobile terminal 600 to execute different operations or process different data according to whether a touch with respect to a point on the touch screen is either a proximity touch or a contact touch.

A touch sensor can sense a touch applied to the touch screen, such as display unit 651, using any of a variety of touch methods. Examples of such touch methods include a resistive type, a capacitive type, an infrared type, and a magnetic field type, among others.

As one example, the touch sensor may be configured to convert changes of pressure applied to a specific part of the display unit 651, or convert capacitance occurring at a specific part of the display unit 651, into electric input signals. The touch sensor may also be configured to sense not only a touched position and a touched area, but also touch pressure and/or touch capacitance. A touch object is generally used to apply a touch input to the touch sensor. Examples of typical touch objects include a finger, a touch pen, a stylus pen, a pointer, or the like.

When a touch input is sensed by a touch sensor, corresponding signals may be transmitted to a touch controller. The touch controller may process the received signals, and then transmit corresponding data to the controller 680. Accordingly, the controller 680 may sense which region on the display unit 651 has been touched. Here, the touch controller may be a component separate from the controller 680, the controller 680, and combinations thereof.

In some embodiments, the controller 680 may execute the same or different controls according to a type of touch object that touches the touch screen or a touch key provided in addition to the touch screen. Whether to execute the same or different control according to the object which provides a touch input may be decided based on a current operating state of the watch-type mobile terminal 600 or a currently executed application program, for example.

The touch sensor and the proximity sensor may be implemented individually, or in combination, to sense various types of touches. Such touches includes a short (or tap) touch, a long touch, a multi-touch, a drag touch, a flick touch, a pinch-in touch, a pinch-out touch, a swipe touch, a hovering touch, and the like.

If desired, an ultrasonic sensor may be implemented to recognize position information relating to a touch object using ultrasonic waves. The controller 680, for example, may calculate a position of a wave generation source based on information sensed by an illumination sensor and a plurality of ultrasonic sensors. Since light is much faster than ultrasonic waves, the time for which the light reaches the optical sensor is much shorter than the time for which the ultrasonic wave reaches the ultrasonic sensor. The position of the wave generation source may be calculated using this fact. For instance, the position of the wave generation source may be calculated using the time difference from the time that the ultrasonic wave reaches the sensor based on the light as a reference signal.

The camera 621 typically includes at least one a camera sensor (CCD, CMOS etc.), a photo sensor (or image sensors), and a laser sensor. Implementing the camera 621 with a laser sensor may allow detection of a touch of a physical object with respect to a 3D stereoscopic image. The photo sensor may be laminated on, or overlapped with, the display device. The photo sensor may be configured to scan movement of the physical object in proximity to the touch screen. In more detail, the photo sensor may include photo diodes and transistors at rows and columns to scan content received at the photo sensor using an electrical signal which changes according to the quantity of applied light. Namely, the photo sensor may calculate the coordinates of the physical object according to variation of light to thus obtain position information of the physical object.

The display unit 651 is generally configured to output information processed in the watch-type mobile terminal 600. For example, the display unit 651 may display execution screen information of an application program executing at the watch-type mobile terminal 600 or user interface (UI) and graphic user interface (GUI) information in response to the execution screen information.

In some embodiments, the display unit 651 may be implemented as a stereoscopic display unit for displaying stereoscopic images. A typical stereoscopic display unit may employ a stereoscopic display scheme such as a stereoscopic scheme (a glass scheme), an auto-stereoscopic scheme (glassless scheme), a projection scheme (holographic scheme), or the like.

The audio output module 652 is generally configured to output audio data. Such audio data may be obtained from any of a number of different sources, such that the audio data may be received from the wireless communication unit 610 or may have been stored in the memory 670. The audio data may be output during modes such as a signal reception mode, a call mode, a record mode, a voice recognition mode, a broadcast reception mode, and the like. The audio output module 652 can provide audible output related to a particular function (e.g., a call signal reception sound, a message reception sound, etc.) performed by the watch-type mobile terminal 600. The audio output module 652 may also be implemented as a receiver, a speaker, a buzzer, or the like.

A haptic module 653 can be configured to generate various tactile effects that a user feels, perceive, or otherwise experience. A typical example of a tactile effect generated by the haptic module 653 is vibration. The strength, pattern and the like of the vibration generated by the haptic module 653 can be controlled by user selection or setting by the controller. For example, the haptic module 653 may output different vibrations in a combining manner or a sequential manner.

Besides vibration, the haptic module 653 can generate various other tactile effects, including an effect by stimulation such as a pin arrangement vertically moving to contact skin, a spray force or suction force of air through a jet orifice or a suction opening, a touch to the skin, a contact of an electrode, electrostatic force, an effect by reproducing the sense of cold and warmth using an element that can absorb or generate heat, and the like.

The haptic module 653 can also be implemented to allow the user to feel a tactile effect through a muscle sensation such as the user's fingers or arm, as well as transferring the tactile effect through direct contact. Two or more haptic modules 653 may be provided according to the particular configuration of the watch-type mobile terminal 600.

An optical output module 654 can output a signal for indicating an event generation using light of a light source. Examples of events generated in the watch-type mobile terminal 600 may include message reception, call signal reception, a missed call, an alarm, a schedule notice, an email reception, information reception through an application, and the like.

A signal output by the optical output module 654 may be implemented in such a manner that the mobile terminal emits monochromatic light or light with a plurality of colors. The signal output may be terminated as the mobile terminal senses that a user has checked the generated event, for example.

The interface unit 660 serves as an interface for external devices to be connected with the watch-type mobile terminal 600. For example, the interface unit 660 can receive data transmitted from an external device, receive power to transfer to elements and components within the watch-type mobile terminal 600, or transmit internal data of the watch-type mobile terminal 600 to such external device. The interface unit 660 may include wired or wireless headset ports, external power supply ports, wired or wireless data ports, memory card ports, ports for connecting a device having an identification module, audio input/output (I/O) ports, video I/O ports, earphone ports, or the like.

The identification module may be a chip that stores various information for authenticating authority of using the watch-type mobile terminal 600 and may include a user identity module (UIM), a subscriber identity module (SIM), a universal subscriber identity module (USIM), and the like. In addition, the device having the identification module (also referred to herein as an “identifying device”) may take the form of a smart card. Accordingly, the identifying device can be connected with the terminal 600 via the interface unit 660.

When the watch-type mobile terminal 600 is connected with an external cradle, the interface unit 660 can serve as a passage to allow power from the cradle to be supplied to the watch-type mobile terminal 600 or may serve as a passage to allow various command signals input by the user from the cradle to be transferred to the mobile terminal there through. Various command signals or power input from the cradle may operate as signals for recognizing that the mobile terminal is properly mounted on the cradle.

The memory 670 can store programs to support operations of the controller 680 and store input/output data (for example, phonebook, messages, still images, videos, etc.). The memory 670 may store data related to various patterns of vibrations and audio which are output in response to touch inputs on the touch screen.

The memory 670 may include one or more types of storage mediums including a Flash memory, a hard disk, a solid state disk, a silicon disk, a multimedia card micro type, a card-type memory (e.g., SD or DX memory, etc), a Random Access Memory (RAM), a Static Random Access Memory (SRAM), a Read-Only Memory (ROM), an Electrically Erasable Programmable Read-Only Memory (EEPROM), a Programmable Read-Only memory (PROM), a magnetic memory, a magnetic disk, an optical disk, and the like. The watch-type mobile terminal 600 may also be operated in relation to a network storage device that performs the storage function of the memory 670 over a network, such as the Internet.

The controller 680 may typically control the general operations of the watch-type mobile terminal 600. For example, the controller 680 may set or release a lock state for restricting a user from inputting a control command with respect to applications when a status of the mobile terminal meets a preset condition.

The controller 680 can also perform the controlling and processing associated with voice calls, data communications, video calls, and the like, or perform pattern recognition processing to recognize a handwriting input or a picture drawing input performed on the touch screen as characters or images, respectively. In addition, the controller 680 can control one or a combination of those components in order to implement various exemplary embodiments disclosed herein.

The power supply unit 690 receives external power or provide internal power and supply the appropriate power required for operating respective elements and components included in the watch-type mobile terminal 600. The power supply unit 690 may include a battery, which is typically rechargeable or be detachably coupled to the terminal body for charging.

The power supply unit 690 may include a connection port. The connection port may be configured as one example of the interface unit 660 to which an external charger for supplying power to recharge the battery is electrically connected.

As another example, the power supply unit 690 may be configured to recharge the battery in a wireless manner without use of the connection port. In this example, the power supply unit 690 can receive power, transferred from an external wireless power transmitter, using at least one of an inductive coupling method which is based on magnetic induction or a magnetic resonance coupling method which is based on electromagnetic resonance.

Various embodiments described herein may be implemented in a computer-readable medium, a machine-readable medium, or similar medium using, for example, software, hardware, or any combination thereof.

As illustrated in FIG. 10B, the watch-type mobile terminal 600 includes a main body 601 with a display unit 651 and a band 604 connected to the main body 401 to be wearable on a wrist.

The main body 601 may include a case having a certain appearance. As illustrated, the case may include a first case 601 a and a second case 601b cooperatively defining an inner space for accommodating various electronic components. Other configurations are possible. For instance, a single case may alternatively be implemented, with such a case being configured to define the inner space, thereby implementing a mobile terminal 600 with a uni-body.

The watch-type mobile terminal 600 can perform wireless communication, and an antenna for the wireless communication can be installed in the main body 601. The antenna may extend its function using the case. For example, a case including a conductive material may be electrically connected to the antenna to extend a ground area or a radiation area.

The display unit 651 is shown located at the front side of the main body 601 so that displayed information is viewable to a user. In some embodiments, the display unit 651 includes a touch sensor so that the display unit can function as a touch screen. As illustrated, window 651a is positioned on the first case 601a to form a front surface of the terminal body together with the first case 601a.

The illustrated embodiment includes audio output module 652, a camera 621, a microphone 622, and a user input unit 623 positioned on the main body 601. When the display unit 651 is implemented as a touch screen, additional function keys may be minimized or eliminated. For example, when the touch screen is implemented, the user input unit 623 may be omitted.

The band 604 is commonly worn on the user's wrist and may be made of a flexible material for facilitating wearing of the device. As one example, the band 604 may be made of fur, rubber, silicon, synthetic resin, or the like. The band 604 may also be configured to be detachable from the main body 601. Accordingly, the band 604 may be replaceable with various types of bands according to a user's preference.

In one configuration, the band 604 may be used for extending the performance of the antenna. For example, the band may include therein a ground extending portion (not shown) electrically connected to the antenna to extend a ground area.

The band 604 may include fastener 603. The fastener 603 may be implemented into a buckle type, a snap-fit hook structure, a Velcro® type, or the like, and include a flexible section or material. The drawing illustrates an example that the fastener 603 is implemented using a buckle.

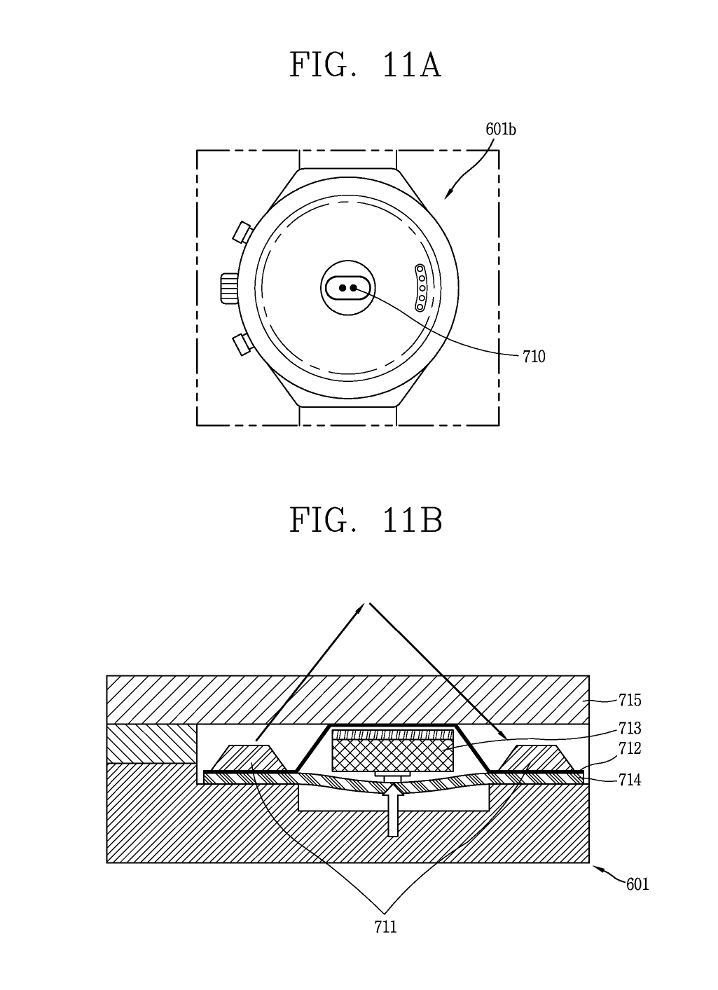

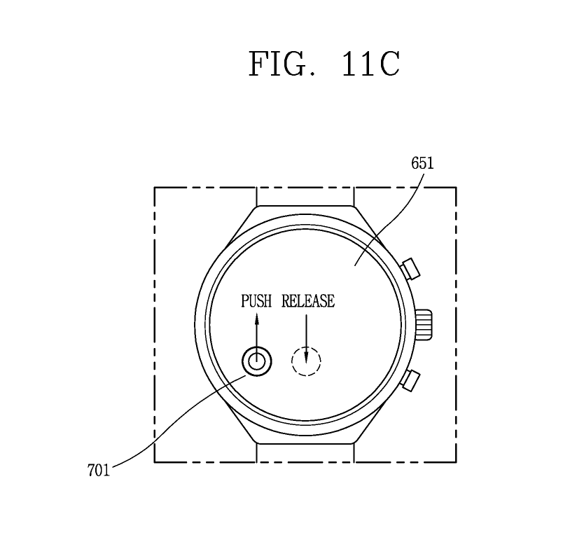

FIG. 11A is a conceptual view illustrating a sensing module for measuring a blood pressure according to an embodiment of the present invention. FIG. 11B is a conceptual view illustrating components of the sensing module. FIG. 11C is a conceptual view illustrating a control method of outputting guide information while a blood pressure is being measured.

The mobile terminal according to this embodiment collects pressure information and pulse wave information by a sensing module 710, and measures a blood pressure based on a periodic open/close state of an artery blood vessel occurring when cuff pressurization and cuff release are executed.

Referring to FIG. 11A, the sensing module 710 according to this embodiment may be disposed such that one region thereof may be exposed to the outside by the second case 601b. The sensing module 710 includes a light emitting portion 711, a light receiving portion 712, a pressure sensor 713, an elastic supporting portion 714 and a window 715.

The light emitting portions 711 and the light receiving portion 712 are covered by the window 715. The light emitting portions 711 may be implemented as green light emitting diodes (LEDs) for emitting green light, but is not limited to this. When a user wears the watch-type mobile terminal 600 on his or her wrist, the window 715 faces the user's wrist. If light emitted from the light emitting portions 711 is reflected from the user's body, the light receiving portion 712 senses the reflected light.

The controller may determine whether the watch-type mobile terminal 600 has been worn or not, based on light sensed by the light receiving portion 712, and may execute a specific function.

The controller may collect pulse wave information based on a pattern of light sensed by the light receiving portion 712. That is, the light emitting portions 711 and the light receiving portion 712 are implemented as a PPG sensor for measuring a pulse wave.

The pressure sensor 713 senses an external force applied from the outside of the mobile terminal. If an external force is applied to the first case 601a or the display unit 651 in a state where the watch-type mobile terminal 600 has been worn on the wrist, a space between the first and second cases 601a, 601b becomes narrow, and the pressure sensor 713 senses the external force. Once the external force is applied, the light emitting portions 711 and the light receiving portion 712 come in contact with the user's body more. As a result, an incident amount of light reflected from the user's skin is changed.

The elastic supporting portion 714 may be implemented as a leaf spring, but is not limited to this. The elastic supporting portion 714 provides an elastic restoration force when the external force is released.

The display unit 651 outputs guide information 701 for guiding a pressure to be applied or released while a pulse wave is being collected. The guide information may be implemented as an image and/or a text instructing a user to apply and release a pressure at specific time intervals.

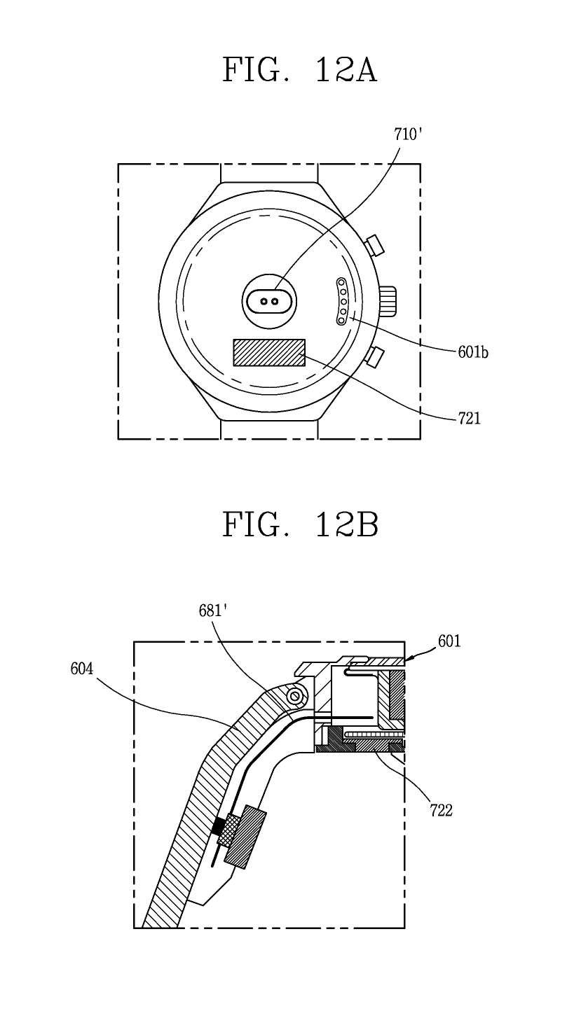

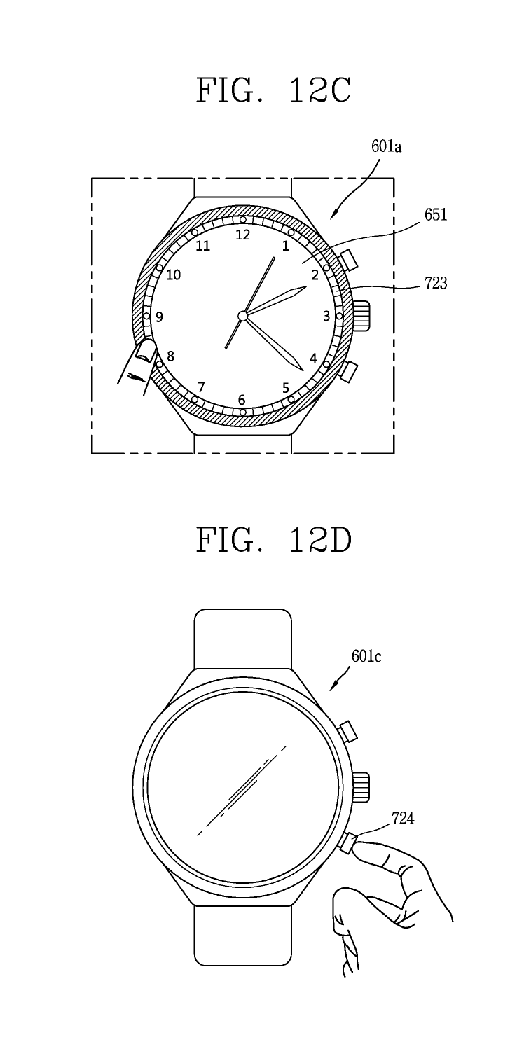

FIGS. 12A to 12D are conceptual views illustrating a mobile terminal including an electrode unit 721 for measuring an electrocardiogram (ECG).

The mobile terminal according to this embodiment may measure a blood pressure based on information on a measured pulse wave and information on a measured electrocardiogram. Hereinafter, will be explained the electrode unit disposed on a specific region of the mobile terminal according to various embodiments of the present invention. The mobile terminal according to various embodiments of the present invention includes a PPG sensor composed of light emitting portions and a light receiving portion so as to measure a pulse wave.

Referring to FIG. 12A, the electrode unit 721 is formed at the second case 601b. The electrode unit 721 may be disposed close to a PPG sensor 710′ composed of a light emitting portion and a light receiving portion. The electrode unit 721 is disposed at the second case 601 b exposed to the outside, and is made to contact a user's skin when the watch-type mobile terminal 600 is worn on the user's wrist.

Referring to FIG. 12B, an electrode unit 722 according to this embodiment is formed on one surface of the band 604. For instance, the electrode unit 722 may be formed on one surface of the terminal body contacting a user's skin when the watch-type mobile terminal 600 is worn. However, the present invention is not limited to this.

The electrode unit 722 is disposed to be exposed to an external surface of the band 604, and the band 604 includes a flexible printed circuit board (FPCB) 681′ for electrically connecting the electrode unit 722 with the body 601. The FPCB 681′ is disposed in the band 604.

Referring to FIG. 12C, an electrode unit 723 according to this embodiment is formed to enclose at least one region of an edge of the display unit 651. That is, the electrode unit 723 is mounted to the first case 601a. In this embodiment, a user may contact his or her body (hand) to the electrode unit 723 more easily, in a worn state of the mobile terminal.

Referring to FIG. 12D, an electrode unit 724 according to this embodiment is formed at a rotatable manipulator 601c for inputting a control command corresponding to a specific function. The rotatable manipulator 601c may be withdrawn to the outside in a connected state to the terminal body, or may be inserted into the terminal body. And the rotatable manipulator 601c may be rotated to two directions. The watch-type mobile terminal 600 may include a plurality of rotatable manipulator.

The mobile terminal according to this embodiment may measure an electrocardiogram when a user's hand contacts the rotatable manipulator 601c in order to execute a specific function.

One electrode unit according to one embodiment may be applied to the mobile terminal together with another electrode unit according to another embodiment. For instance, the electrode units shown in FIGS. 12A and 12D may be included in a single mobile terminal.

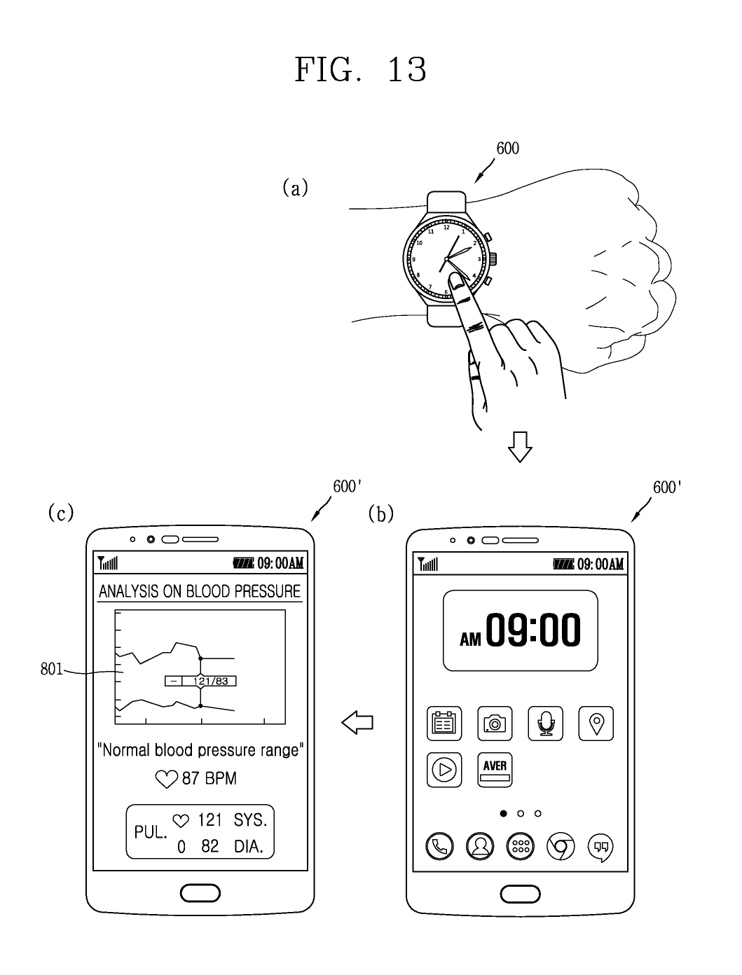

FIG. 13 is a conceptual view illustrating a control method of providing a result on a blood pressure measured according to an embodiment of the present invention.

Referring to FIGS. 12A, 12C and 13, a watch-type mobile terminal 600 according to this embodiment includes a PPG sensor, a first electrode unit mounted to the second case 601b, and a second electrode unit mounted to the first case 601a.

A controller 680 collects electrocardiogram information when a finger contacts the second electrode unit, in a state where the watch-type mobile terminal 600 has been worn on a user's wrist. Once the electrocardiogram information and pulse wave information by the PPG sensor are collected, the controller calculates a blood pressure. The controller 680 may transmit information on the calculated blood pressure, to an external device 600′ wirelessly connected to the mobile terminal. Alternatively, the controller 680 may transmit the collected electrocardiogram information and pulse wave information, to the external device 600′.

Once the blood pressure information is received, the external device 600′ may output a result screen 801 including the blood pressure information to the display unit.

The external terminal may be implemented using a variety of different types of terminals. Examples of such terminals include cellular phones, smart phones, user equipment, laptop computers, digital broadcast terminals, personal digital assistants (PDAs), portable multimedia players (PMPs), navigators, portable computers (PCs), slate PCs, tablet PCs, ultra books, wearable devices (for example, smart watches, smart glasses, head mounted displays (HMDs)), and the like.

Various embodiments may be implemented using a machine-readable medium having instructions stored thereon for execution by a processor to perform various methods presented herein. Examples of possible machine-readable mediums include HDD(Hard Disk Drive), SSD(Solid State Disk), SDD(Silicon Disk Drive), ROM, RAM, CD-ROM, a magnetic tape, a floppy disk, an optical data storage device, the other types of storage mediums presented herein, and combinations thereof. If desired, the machine-readable medium may be realized in the form of a carrier wave (for example, a transmission over the Internet). The processor may include the controller 680 of the mobile terminal.

### INDUSTRIAL APPLICABILITY

Various embodiments of the present invention provide a mobile terminal capable of measuring a blood pressure based on bio information, and the mobile terminal may be applied to various industrial fields.

1. A mobile terminal, comprising:
a terminal body;
a first sensor part disposed at an external side of the terminal body, the first sensor part comprising a light emitting portion and a light receiving portion and configured to collect pulse wave information;
a second sensor part disposed at one region of the terminal body and configured to collect an additional biosignal; and
a controller configured to calculate a blood pressure based on the pulse wave information and the additional biosignal.

2. The mobile terminal of claim 1, wherein:
the second sensor part corresponds to a pressure sensor for sensing an external force, and
the additional biosignal corresponds to a pressure change sensed while the pulse wave information is collected by the first sensor part.

3. The mobile terminal of claim 2, wherein the second sensor part comprises an actuator configured to be moveable by pressure to cause a control signal.

4. The mobile terminal of claim 2, wherein:
the pressure sensor is disposed in the terminal body, and
the terminal body further comprises an elastic supporting portion for providing a restoration force corresponding to the external force.

5. The mobile terminal of claim 1, wherein:
the second sensor part comprises first and second electrode portions disposed on different regions of the terminal body and configured to generate a potential difference, and
the controller is further configured to collect electrocardiogram information based on the potential difference.

6. The mobile terminal of claim 5, wherein the second electrode portion comprises a metallic member disposed proximate to the first sensor part.

7. The mobile terminal of claim 6, wherein:
the terminal body comprises an electronic component having one or more externally exposed regions, and
the metallic member encloses one region of the electronic component.

8. The mobile terminal of claim 7, wherein the electronic component corresponds to:
a signal input unit for inputting a control command,
a camera for capturing an image,
an audio output unit for outputting an audio signal, or
a sensing module for emitting laser for an auto focusing function.

9. The mobile terminal of claim 8, wherein the signal input unit comprises a rotatable manipulator configured to be inserted, withdrawn, or rotated in a mounted state to the terminal body to cause execution of a specific function.

10. The mobile terminal of claim 6, further comprising a band for fixing the terminal body to a user's wrist, wherein:
the second electrode portion is disposed on one region of the band, and
the band comprises a flexible printed circuit board configured to electrically connect the second electrode portion to the terminal body.

11. The mobile terminal of claim 7, wherein the metallic member of the second electrode portion is disposed at a side corresponding to the one region of the terminal body.

12. The mobile terminal of claim 7, wherein:
the second sensor part includes a transmission region for transmitting an electric signal and a receiving region for receiving the electric signal reflected from a finger for sensing a fingerprint, and
the second electrode portion corresponds to the receiving region.

13. The mobile terminal of claim 1, further comprising a camera, wherein:
the controller is further configured to control a focal point of the camera based on laser which is emitted by the first sensor part, reflected, and incident onto the light receiving portion.

14. The mobile terminal of claim 1, further comprising a wireless communication unit configured to perform wireless communication with an external device, wherein:
the second sensor part corresponds to a first electrode portion of a metallic member externally exposed at the terminal body, and
the controller is further configured to collect electrocardiogram information based on a potential difference between a second electrode portion of the external device and the first electrode portion.
